(19) United States
(12) Patent Application Publication
(10) Pub. No.: US 2023/0352120 A1
(43) Pub. Date: Nov. 2, 2023

(54) SYSTEM AND METHOD FOR PREDICTING EFFICIENCY AND OUTCOME OF BASE EDITOR BY USING DEEP LEARNING

(71) Applicant: INDUSTRY-ACADEMIC COOPERATION FOUNDATION, YONSEI UNIVERSITY, Seoul (KR)

(72) Inventors: Hyongbum Henry KIM, Seoul (KR); Nahye KIM, Tongyeong-si (KR)

(73) Assignee: INDUSTRY-ACADEMIC COOPERATION FOUNDATION, YONSEI UNIVERSITY, Seoul (KR)

(21) Appl. No.: 18/309,208

(22) Filed: Apr. 28, 2023

(30) Foreign Application Priority Data
Apr. 29, 2022 (KR) 10-2022-0053742
Apr. 27, 2023 (KR) 10-2023-0055651

(51) Int. Cl. G16B 40/00; G16B 30/00

(52) U.S. Cl. CPC G16B 40/00; G16B 30/00

(57) ABSTRACT

According to a system for predicting the efficiency and an outcome of a base editor by using deep learning, it is possible to select a base editor from among 63 base editors with various protospacer adjacent motif (PAM) compatibilities and sgRNA for efficient base editing, without extensive experiments. Therefore, the system may be usefully used in all fields where gene editing is applied, such as disease treatment by gene editing.

### CROSS-REFERENCE TO RELATED APPLICATION

This application is based on and claims priority under 35 U.S.C. § 119 to Korean Patent Application No. 10-2022-0053742, filed on Apr. 29, 2022, in the Korean Intellectual Property Office, and Korean Patent Application No. 10-2023-0055651, filed on Apr. 27, 2023, in the Korean Intellectual Property Office, the disclosure of which is incorporated by reference herein in its entirety.

### BACKGROUND

### 1. Field

The disclosure relates to a system for predicting the efficiency and an outcome of a base editor by using deep learning.

### 2. Description of the Related Art

Base editing enables the conversion of one base pair to another without requiring donor DNA or generating double-strand breaks. A base editor is composed of a base editor protein and a single-guide RNA (sgRNA). Base editor proteins are essentially fusions of Cas9 nickase and base-modifying enzymes such as cytidine or adenosine deaminases. Cytosine base editors (CBEs) can convert C•G to T•A and adenine base editors (ABEs) can convert A•T to G•C. In the case of CBEs, uracil glycosylase inhibitor (UGI) is frequently added to enhance the base editing efficiencies and purities. In addition to these two major classes of base editors, C•G to G•C base editors (CGBEs), which are derived from CBEs by the removal of UGI and/or the addition of uracil DNA N-glycosylase (UNG), can convert C•G to G•C. To improve the efficiency and fidelity of base editing, base editors with improved base-converting domains (i.e., deaminase with or without assisting factors such as UGI or UNG) have been developed: YE1-BE4max, SsAPOBEC3B, ABE8e(V106W), ABE8.17-m+V106W, CGBE1, miniCGBE1, and APOBEC-nCas9-Ung. However, the choice of which base-converting domain variant-containing base editor to use is confusing because these variants have not been extensively compared.

In addition to the base-converting domain, another variable in base editing is the Cas9 nickase, which recognizes a target sequence containing a protospacer-adjacent motif (PAM) that is located ˜15±2 nucleotides from the target nucleotide. The canonical PAM sequence for SpCas9 is NGG and this PAM is often not available at the desired position, blocking efficient base editing with minimal bystander editing. This PAM requirement also often limits applications of Cas9 for other types of genome editing (e.g., performing tiling screening, generating targeted deletions, and obtaining efficient homology-directed genetic modifications) as well as base editing. To overcome these restrictions, Cas9 variants with different PAM compatibilities have been developed. Although we previously performed extensive comparisons of some of the early versions of Cas9 variants that recognize non-NGG PAMs such as xCas9, SpCas9-NG, and the VQR, VRER, VRQR, and QQR1 variants, more variants have been reported since our study: SpCas9-NRRH, SpCas9-NRTH, SpCas9-NRCH, SpG, SpRY, and Sc++. Because these variants have not been extensively compared, the choice of which Cas9 to use at a given target sequence can be difficult, especially for base editing.

The inventors of the present invention extensively compared seven base editor variants with different base-converting domains (YE1-BE4max, SsAPOBEC3B, ABE8e(V106W), ABE8.17-m+V106W, CGBE1, miniCGBE1, and APOBEC-nCas9-Ung) (FIG. 1a), ten Cas9 variants with different or altered PAM compatibilities (VRQR variant, xCas9, SpCas9-NG, SpCas9-NRRH, SpCas9-NRTH, SpCas9-NRCH, SpG, SpRY, and Sc++) (FIG. 1b), and ten base editor variants containing Cas9 nickase domains with different or altered PAM compatibilities (SpCas9-YE1-BE4max, SpCas9-NRCH-YE1-BE4max, SpRY-YE1-BE4max, SpCas9-NRCH-SsAPOBEC3B, SpCas9-ABE8e(V106W), SpRY-ABE8e(V106W), SpCas9-NRCH-ABE8.17-m+V106W, SpRY-ABE8.17-m+V106W, SpCas9-miniCGBE1, and SpCas9-NRCH-APOBEC-nCas9-Ung) (FIG. 1c) at hundreds or thousands of target sequences. Based on the resulting large-sized data sets of Cas9 and base editor variant activities, we developed DeepCas9variants, a deep learning-based computational model that predicts the efficiency of the nine Cas9 variants, and DeepBE, which predicts the efficiency and editing outcome frequencies of the 63 base editors generated by combining the seven types of deaminating domains with the nine Cas9 variants.

### SUMMARY

Provided is a system for predicting the efficiency and an outcome of a base editor by using deep learning.

Provided is a method of predicting the efficiency and an outcome of a base editor by using deep learning.

Provided is a computer-readable recording medium having recorded thereon a program for causing a computer to execute a method of predicting the efficiency and an outcome of a base editor by using deep learning.

Additional aspects will be set forth in part in the description which follows and, in part, will be apparent from the description, or may be learned by practice of the presented embodiments of the disclosure.

Provided is a system for predicting the efficiency and an outcome of a base editor by using deep learning.

In detail, provided is a system for predicting the efficiency and an outcome of a base editor by using deep learning, the system including a target sequence input unit configured to receive an input of target sequence data of the base editor, and an outcome prediction unit configured to obtain a base editing efficiency output value and a base editing outcome proportion output value by applying the target sequence data that is input through the target sequence input unit, to a base editing efficiency prediction model and a base editing outcome proportion prediction model, respectively, and generate a base editing prediction score by multiplying the base editing efficiency output value by the base editing outcome proportion output value.

As used herein, the term “base editor (BE)” refers to a new type of genome editor derived from the CRISPR base editor, which is called the fourth generation genome editing technology, and works by converting a single base. In detail, the BE is composed of a base editor protein and a single-guide RNA (sgRNA), and the base editor protein is a fusion of Cas9 nickase and base-modifying enzymes such as cytidine or adenosine deaminases. Representative examples of BEs include adenine BEs (ABEs), which are obtained by fusing an adenine deaminase with dCas9 (“dead” Cas9) or nCas9 without the double-stranded DNA cleavage function of CRISPR/Cas9, and may convert A•T to G•C, cytosine BEs (CBEs), which are obtained by fusing a cytosine deaminase with dCas9 or nCas9 without the double-stranded DNA cleavage function of CRISPR/Cas9, and may convert C•G to T•A, and C•G to G•C BEs (CGBEs) that may convert C•G to G•C. For example, the CBEs work on the principle that, when a deaminase converts cytosine (C) to uracil (U) in one strand of DNA cleaved by nCas9 or dCas9, the base that has undergone the conversion to uracil (U) is converted to thymine (T) by a DNA repair process. By using such BEs, a gene may be deleted or converted into a desired trait by correcting or converting a particular sequence.

In detail, the BE may be any one or more selected from the group consisting of YE1-BE4max, SsAPOBEC3B, ABE8e(V106W), ABE8.17-m+V106W, CGBE1, miniCGBE1, and APOBEC-nCas9-Ung, but is not particularly limited.

As used herein, the term “guide RNA” refers to an RNA that is specific to a target DNA sequence, and may complementarily bind to all or part of a target sequence such that an adenine deaminase or a cytosine deaminase of a base editor finds adenine (A) or cytosine (C) in the target sequence and converts the adenine (A) or the cytosine (C) to guanine (G) or thymine (T), respectively.

In general, the guide RNA refers to a dual RNA including a single-guide RNA (sgRNA), CRISPR RNA (crRNA), trans-activating crRNA (tracrRNA)) as constituting elements, or refers to a form that includes a first region including a sequence complementary to all or part of a sequence in a target DNA, and a second region including a sequence interacting with an RNA-guided nuclease, but any form where an RNA-guided nuclease may have activity in a target sequence may be included in the scope of the disclosure without limitation. In addition, the guide RNA may include a scaffold sequence which helps the attachment of an RNA-guided nuclease.

As used herein, the term “Cas9 protein” refers to a major protein element of the CRISPR/Cas9 system, and the Cas9 protein forms a complex with CRISPR RNA (crRNA) and trans-activating crRNA (tracrRNA) to form activated endonuclease or nickase. Information about the Cas9 protein or genes thereof may be obtained from a known database such as GenBank of National Center for Biotechnology Information (NCBI), but any Cas9 protein having target-specific nuclease activity together with guide RNA may be included in the scope of the disclosure. In addition, the Cas9 protein may be bound with a protein transduction domain. The protein transduction domain may be poly-arginine or HIV T•A•T protein, but is not limited thereto. Furthermore, an additional domain may be suitably bound to the Cas9 protein by those skill in the art according to the intended use.

The Cas9 protein may include not only wild-type Cas9, but also deactivated Cas9 (dCas9), or Cas9 variants such as Cas9 nickase. The deactivated Cas9 may be RNA-guided FokI nuclease (RFN) including a FokI nuclease domain bound to dCas9, or may be dCas9 to which a transcription activator or repressor domain is bound, and the Cas9 nickase may be D10A Cas9 or H840A Cas9, but is not limited thereto. In detail, the Cas9 may be any one or more selected from the group consisting of SpCas9, VRQR variant, SpCas9-NG, SpCas9-NRRH, SpCas9-NRTH, SpCas9-NRCH, SpG, SpRY, and Sc++.

The Cas9 protein is not limited in its origin. For example, the Cas9 protein may be derived from Streptococcus pyogenes, Francisella novicida, Streptococcus thermophilus, Legionella pneumophila, Listeria innocua, or Streptococcus mutans.

As used herein, the term “target sequence” refers to a nucleotide sequence expected to be targeted by a BE. In detail, the target sequence is a sequence that a BE is expected to target through a guide RNA, and may be a known sequence on which the BE exhibits an activity, or may be a sequence arbitrarily designed based on a sequence that one of skill in the art using the system of the disclosure to analyze, but any sequence that is to be analyzed as the BE exhibits or is expected to exhibit an activity thereon may be included in the scope of the disclosure without limitation.

In the present specification, base conversion activity data of the BE may be obtained by introducing the BE into a cell library containing oligonucleotides including a nucleotide sequence that encodes sgRNA and a target nucleotide sequence targeted by the sgRNA, and the disclosure is not limited thereto.

As used herein, the term “target sequence input unit” refers to a component that is included in a system for predicting the efficiency and an outcome of a BE by using deep learning, and is configured to receive an input of the target sequence.

As used herein, the term “activity” or “efficiency” of a BE refers to an activity of the BE by which a single base is converted, that is, for example, an activity that causes a RNA-guided nuclease, particularly, Cas9, to cleave genes, and causes a deaminase to convert adenine (A) to guanine (G) or cytosine (C) to thymine (T). As used herein, the term “activity data” corresponds to data for extracting and learning the relationship between a particular input sequence or a target sequence and the BE, and the system of the disclosure may generate a base editing efficiency prediction model by using the activity data.

In detail, the activity data of the BE may be obtained by performing sequence analysis on bases of a target sequence. For example, deep sequencing, RNAseq, or the like may be performed to obtain resulting data, but any method may be used as long as it is possible to obtain activity data of a BE through detection of edited bases. The activity data of the BE may be existing known activity data, or may be activity data directly obtained by any method that may be appropriately adopted by one of skill in the art, and for the purpose of the disclosure, any method of obtaining data may be used as long as data for generating an activity prediction model capable of predicting the activity of a BE is obtained.

As used herein, the term “base editing outcome” of a BE refers to an editing product generated as a result of an activity of the BE on a target sequence. Meanwhile, in a case in which there are a plurality of editable target nucleotides within a base editing range (editable window), an unwanted base may be edited, and as used herein, the term “base editing frequency” refers to the frequency of each product produced as a result of the activity of the BE.

The base editing efficiency prediction model may be generated by: receiving an input of base conversion activity data of a BE through an information input unit; and generating the base editing efficiency prediction model by performing deep learning based on a convolutional neural network (CNN) on the data input through the information input unit.

As used herein, the term “information input unit” refers to a component configured to receive base conversion activity data or base editing outcome data of a BE, and the information input unit may directly receive, from a user of a prediction system according to an embodiment, an input data about the BE, or may receive an input of pre-stored data, but is not limited thereto.

An output value of the base editing efficiency may be calculated through Equation 1 below.

$$Base editing efficiency ( \% ) = \frac{\begin{matrix}Total read counts of intended target \\ nucleotide conversions at each position\end{matrix}}{Total read counts} \times 100 \qquad [ Equation 1 ]$$

In addition, an output value of the base editing outcome proportion may be calculated through Equation 2 below.

$$Base editing outcome proportion = \frac{\begin{matrix}Total read counts of unique \\ base - edited outcome sequence\end{matrix}}{\begin{matrix}Total read counts of converted \\ sequences within wide windows\end{matrix}} \qquad [ Equation 2 ]$$

As used herein, the term “deep learning” refers to artificial intelligence (AI) technology that allows computers to think and learn like humans, and allows machines to learn and solve complex nonlinear problems on their own based on the artificial neural network theory. By using deep learning technology, it is possible to enable computers to recognize, infer, and judge on their own even when humans do not set all criteria for judgement, and thus to be widely used for voice and image recognition, image analysis, and the like. In other words, deep learning may be defined as a set of machine learning algorithms that attempt high-level abstractions (summarizing key content or functions in large amounts of data or complex materials) through a combination of several nonlinear transformation methods.

As used herein, the term “convolutional neural network (CNN)” refers to a technique of extracting features representing a part of provided information and achieving generalization through hierarchization of information.

The generating of the base editing efficiency prediction model by performing the deep learning based on the CNN may further include linking CRISPR associated protein 9 (Cas9) activity data, and the CAS9 activity data may be linked to a flatten layer of the system for predicting the efficiency and an outcome of a BE.

In addition, the Cas9 activity data may be obtained by performing a method including: introducing Cas9 into a cell library containing oligonucleotides including a nucleotide sequence that encodes sgRNA and a target nucleotide sequence targeted by the sgRNA; performing deep sequencing by using DNA obtained from the cell library into which the Cas9 is introduced; and analyzing the efficiency of the Cas9 based on data obtained from the deep sequencing, and the Cas9 activity data may be generated or output in the form of prediction scores.

As used herein, the term “library” refers to a pool or population including two or more types of substances wherein the substances of the same type have different characteristics. Thus, an oligonucleotide library may be a population including two or more types of oligonucleotides having different base sequences, for example, two types of oligonucleotides having different guide RNAs and/or target sequences, and a cell library may be a population of two or more cells with different characteristics, particularly, a population of cells having different oligonucleotides included therein for the purpose of the disclosure, for example, a population of cells having different guide RNA introduced therein and/or target sequences or types.

As used herein, the term “vector” refers to a medium or a genetic construct that allows the oligonucleotide to be delivered into a cell, and a vector herein may include an oligonucleotide containing each guide RNA-coding sequence and a target sequence. The vector may be a viral vector or a plasmid vector, and the viral vector may be specifically a lentiviral vector, a retroviral vector, or the like, but is not limited thereto, and those of skill in the art may freely use a known vector as long as it may achieve the objective of the disclosure. In detail, the vector may contain essential regulatory elements operably linked to an insert, that is, the oligonucleotide, such that the oligonucleotide may be expressed when the vector is present in a cell of a subject.

The vector may be prepared and purified by using standard recombinant DNA techniques. The type of the vector is not particularly limited as long as it may act in target cells such as prokaryotic cells and eukaryotic cells. In addition, the vector may include a promoter, an initiation codon, and a stop codon terminator, and in addition, may also appropriately include DNA that codes a signal peptide, an enhancer sequence, a 5′ or 3′ untranslated region, a selection marker region, and/or a replicable unit.

A method of delivering the vector to a cell for preparing a library may be achieved by using various methods known in the art. These methods may include, for example, calcium phosphate-DNA co-precipitation method, a DEAE-dextran-mediated transfection method, polybrene-mediated transfection method, electroporation, microinjection, liposome fusion method, Lipofectamine and protoplast fusion method, etc. which are known in the art. In addition, when a viral vector is used, a target product, that is, a vector, may be delivered by using virus particles having the infection as a means. Furthermore, the vector may be introduced into a cell by gene bombardment, etc. The introduced vector may be present as a vector itself in the cell or may be integrated into the chromosome, but the disclosure is not limited thereto.

The analyzing of the efficiency of the Cas9 may be to predict the activity of the Cas9 based on a correlation between indel frequencies of the Cas9 in a particular target sequence by performing deep learning based on a CNN.

The base editing outcome proportion prediction model may be generated by: receiving base editing outcome data of a BE through an information input unit; and generating the base editing outcome proportion prediction model by performing deep learning based on a CNN on the data input through the information input unit. The descriptions provided above are also applied to the base editing outcome proportion prediction model. The term “outcome data” corresponds to data for extracting and learning the relationship between a particular input sequence or a target sequence and the BE, and the system of the disclosure may generate a base editing outcome proportion model by using the outcome data.

As used herein, the term “outcome prediction unit” refers to a component configured to predict the base editing efficiency and an outcome of a BE by applying a target sequence that is input through a target sequence input unit to a base editing efficiency prediction model and a base editing outcome proportion prediction model. In an embodiment, the outcome prediction unit may predict the base editing efficiency and an outcome proportion of the BE from target sequence information.

The system may further include an output unit configured to output the efficiency and an outcome proportion of the BE predicted by the outcome prediction unit. In addition, the prediction system of the disclosure may further include a storage unit storing previously obtained data about a BE or data about a known BE, and in a case in which the prediction system includes the storage unit, the information input unit of the prediction system of the disclosure may receive data of a set size or range from the storage unit, and use the data to predict the base editing efficiency and an editing outcome proportion of the BE.

Provided a method of predicting the efficiency and an outcome of a BE by using deep learning. In detail, provided is a method of predicting the efficiency and an outcome of a BE by using deep learning including: designing a target sequence of the BE; and applying the designed target sequence to the system for predicting the efficiency and an outcome of a BE. The descriptions provided above are also applied to the method of predicting the efficiency and an outcome of a BE.

Provided is a computer-readable recording medium having recorded thereon a program for causing a computer to execute a method of predicting the efficiency and an outcome of a BE by using deep learning.

The program may be an implementation of the system for predicting the efficiency and an outcome of a BE or the method of predicting the efficiency and an outcome of a BE according to an aspect in a computer programming language.

Computer programming languages capable of implementing the program of the disclosure include Python, C, C++, Java, Fortran, Visual Basic, and the like, but are not limited thereto. The program may be stored in a recording medium such as a Universal Serial Bus (USB) memory, a compact disc read-only memory (CD-ROM), a hard disk, a magnetic diskette, or a similar medium or device, and may be connected to an internal or external network system. For example, a computer system may access a sequence database such as GenBank (http://www.ncbi nlm.nih.gov/nucleotide) by using Hypertext Transfer Protocol (HTTP), Hypertext Transfer Protocol Secure (HTTPS), or Extensible Markup Language (XML) protocols, to search for a target gene and the nucleic acid sequence of the regulatory region of the target gene.

The program may be provided online or offline, and may be provided in the form of a computer program stored in a recording medium to execute the system for predicting the efficiency and an outcome of a BE in combination with a computer-implemented electronic device.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1C show base editors and Cas9 variants that have been experimentally evaluated in the disclosure, and the red arrows indicate introduced mutations; FIG. 1A shows base editor variants containing an SpCas9-NG nickase region and several deaminase regions, FIG. 1B shows Cas9 variants having different protospacer adjacent motif (PAM) compatibilities, and FIG. 1C shows base editor variants containing Cas9 domains with different PAM compatibilities;

FIG. 2 shows comparison of indel frequencies measured by using two slightly different bulk evaluation methods, and correlations of indel frequencies of two biological replicates; a of FIG. 2 shows a comparison of indel frequencies measured before (x-axis) and after (y-axis) removal of sequencing reads containing errors or shuffled sequences, wherein the numbers of target sequences for SpCas9, VRQR variant, xCas9 and SpCas9-NG are n=11,668, 11,678, 11,661 and 11,659, respectively; b of FIG. 2 shows correlations of indel frequencies between two biological replicates, for which two independent transductions of lentiviral library A were performed with two independently generated SpCas9-expressing cell populations or an SpCas9-NRCH-expressing cell population on another day; For SpCas9 and SpCas9-NRCH, the numbers of target sequences are (n)=11,680 and 11,590, respectively;

FIGS. 3A and 3B are scatter plots showing Cas9 variant-induced indel frequencies evaluated by using libraries A and B, and FIGS. 3C, 3D, 3E, 3F, and 3G are scatter plots each showing base conversions induced by a base editor, and base conversion efficiencies mediated by SpCas9-YE1-BE4max (FIG. 3C) and SpCas9-ABE8e (V106W) (FIG. 3D) in libraries A and B, or mediated by CBE variants (FIG. 3E), ABE variants (FIG. 3F), and CGBE variants (FIG. 3G) including Cas9 variants;

FIGS. 4A to 4G show efficiencies of base conversions indicated at respective positions on target sequences for SpCas9-NG-YE1-BE4max (FIG. 4A), SpCas9-NG-SsAPOBEC3B (FIG. 4B), SpCas9-NG-ABE8e (V106W) (FIG. 4C), SpCas9-NG-ABE8.17-m+V106W (FIG. 4D), SpCas9-NG-CGBE1 (FIG. 4E), SpCas9-NG-miniCGBE1 (FIG. 4F), and SpCas9-NG-APOBEC-nCas9-Ung (FIG. 4G), for which target sequences containing NG PAMs were analyzed;

FIG. 5 shows preferred motifs for intended base conversions induced by SpCas9-NG-YE1-BE4max (a of FIG. 5) and SpCas9-NG-SsAPOBEC3B (b of FIG. 5), wherein the heat maps show the dependence of the average editing efficiency at a desired base on the neighboring nucleotides (target sequences with NG PAMs were used);

FIG. 6 shows preferred motifs for SpCas9-NG-induced indel frequencies at position 6, and the heat maps show the dependence of SpCas9-NG-induced indel frequencies on nucleotides adjacent to a target nucleotide;

FIG. 7 shows preferred motifs for intended base conversions induced by SpCas9-NG-ABE8e(V106W) (a of FIG. 7) and SpCas9-NG-ABE8.17-m+V106W (b of FIG. 7), wherein the heat maps show the dependence of the average editing efficiency at a desired base on the neighboring nucleotides (target sequences with NG PAMs were used);

FIG. 8 shows preferred motifs for intended base conversions induced by SpCas9-NG-CGBE1 (a of FIG. 8), SpCas9-NG-miniCGBE1 (b of FIG. 8), and SpCas9-NG-APOBEC-nCas9-Ung (c of FIG. 8), wherein the heat maps show the dependence of the average editing efficiency at a desired base on the neighboring nucleotides (target sequences with NG PAMs were used);

FIG. 9 shows the dependence of conversion efficiencies from C•G to G•C or from C•G to T•A (a to c of FIG. 9) for SpCas9-NG-CGBE1 (a and d of FIG. 9), SpCas9-NG-miniCGBE1 (b and e of FIG. 9), and SpCas9-NG-APOBEC-nCas9-Ung (c and f of FIG. 9), and product purities (d to f of FIG. 9) of conversions from C•G to G•C at position 6 on neighboring nucleotides (target sequences with NG PAMs were used);

FIGS. 10A to 10K show correlations between average Cas9 variant-induced indel frequencies at target sequences containing each 4-nt PAM sequence, and average base editing conversion efficiencies mediated by base editor variants containing the corresponding Cas9 variants, wherein the ranks of average indel frequencies are shown (FIGS. 10B to 10K);

FIG. 11 shows base editing efficiencies induced by base editor variants containing Cas9 variants as nickase regions at respective positions of target sequences;

FIG. 12 shows effects of nucleotides adjacent to target nucleotides on Cas9 variant-containing base editors, wherein the heat maps show the dependence of average efficiencies of intended base conversions on neighboring nucleotides, at ABE8e (V106W) based on Cas9 variants such as SpCas9, SpCas9-NG, and SpRY (a of FIG. 12), ABE8.17-m+V106W (b of FIG. 12), miniCGBE1 (c of FIG. 12), and APOBEC-nCas9-Ung (d of FIG. 12);

FIG. 13 shows activities of SpCas9 PAM variants, wherein a of FIG. 13 shows indel frequencies in 23 target sequences that perfectly match an NGG PAM used to calculate specificities, the number of target sequences is (n)=23, and different target sequences are distinguished by different colors; b of FIG. 13 shows relative indel frequencies induced by respective Cas9 variants based on a single-base mismatch type;

FIG. 14 shows relative indel frequencies induced by SpCas9 PAM variants in target sequences containing consecutive two-base mismatches at sites with NGG PAMs;

FIG. 15 shows relative indel frequencies induced by SpCas9 PAM variants in target sequences containing consecutive three-base mismatches at sites with NGG PAMs;

FIG. 16 shows algorithms used to develop DeepCas9variants (a of FIG. 16) and DeepNG-BE (b of FIG. 16);

FIG. 17 shows an editing window, product purity, and preferred motifs for CBE and ABE variants, for which base editors containing SpCas9-NG were evaluated in target sequences with NG PAMs;

FIG. 18 shows an editing window, product purity, and preferred motifs for CGBE variants; a of FIG. 18 shows conversion efficiencies from cytosine to guanine at respective positions of target sequences, b of FIG. 18 shows product purities associated with base editor variants, and c of FIG. 18 shows preferred motifs for base editing, wherein the heat maps show the dependence of the average editing efficiency at a desired base on neighboring nucleotides; d to f of FIG. 18 show comparison of base editing efficiencies induced by various base editors, wherein the red triangles indicate target sequences at which the editing efficiency of one base editor is at least 30% higher than that of the other base editor;

FIG. 19 shows comparison of Cas9 variants with different PAM compatibilities and integration of the variants as nickase domains for base editors; a and b of FIG. 19 show maximum average indel frequencies generated by one of ten Cas9 variants (a of FIG. 19), and the corresponding Cas9 variants that show the highest average activities (b of FIG. 19); c of FIG. 19 shows correlations between indel frequencies induced by Cas9 variants at target sequences with 4-nt PAM sequences, d of FIG. 9 shows a percentages of guide sequences for which a preferred Cas9 variant at a given PAM sequence shows lower activity than one of the remaining Cas9 variants at sites with the PAM by at least 1.3-fold, e of FIG. 19 shows comparison of average indel frequencies and base editing efficiencies at target sequences with the indicated shared 2-nt PAM sequences, and f of FIG. 19 shows correlations between average indel frequencies induced by SpCas9-NRCH and average base editing efficiencies mediated by base editor variants containing SpCas9-NRCH at targets with 4-nt PAM sequences; g and h of FIG. 19 show effects of nucleotides adjacent to a target nucleotide on base editors based on Cas9 variants, wherein the heat maps show the dependence of average efficiencies of intended base conversions on neighboring nucleotides for YE1-BE4max (g of FIG. 19) and SsAPOBEC3B (h of FIG. 19) containing Cas9 variants such as SpCas9, SpCas9-NG, and SpCas9-NRCH;

FIG. 20 shows specificity of SpCas9-YE1-BE4max, SpCas9-ABE8e(V106W), and SpCas9 variants with different PAM compatibilities; a of FIG. 20 is a heat map showing the specificity of the SpCas9 variants at target sequences with single-base mismatches compared to perfectly matched target sequences, and b and c of FIG. 20 show relative indel frequencies at sites containing consecutive two-base (b of FIG. 20) and three-base (c of FIG. 20) mismatches at positions 1 to 20; d and e of FIG. 20 are heat maps showing the specificity of SpCas9-YE1-BE4max (d of FIG. 20) and SpCas9-ABE8e(V106W) (e of FIG. 20), and f and g of FIG. 20 show dependence of relative base editing efficiencies of SpCas9-YE1-BE4max (f of FIG. 20) and SpCas9-ABE8e(V106W) (g of FIG. 20) on the type of single-base mismatch; h and i of FIG. 20 show effects of two-base (h of FIG. 20) and three-base (i of FIG. 20) consecutive mismatches on base editing;

FIG. 21 shows deep learning-based prediction of activities of Cas9 nuclease variants and base editor variants; a of FIG. 21 is a schematic representation of an algorithm used to develop computational models, and b to d of FIG. 21 show correlations between predicted and measured indel frequencies induced by Cas9 variants (b of FIG. 21) and base editing frequencies induced by base editor variants containing Cas9 variants (c to d of FIG. 21) at target sequences in a held-out test data set; e of FIG. 21 shows results of evaluating the performance of DeepBE by using test data sets that were generated by using base editor variants, and were not used for training;

FIG. 22 shows correction of pathogenic or likely pathogenic single-nucleotide polymorphisms (SNPs) using base editors, and shows total editing efficiencies and bystander-free intended editing efficiencies of SNP corrections induced by CBEs (a of FIG. 22), ABEs (b of FIG. 22), and CGBEs (c of FIG. 22);

FIG. 23 shows generation and evaluation of variant-expressing cell lines; a of FIG. 23 shows a schematic overview of a library experiment, and b of FIG. 23 shows western blot analysis of Cas9 protein levels in cells expressing Cas9 variants, base editor variants with different deaminases, and base editor variants containing Cas9 variants;

FIG. 24 shows comparison of base editing efficiencies induced by different CBEs (a of FIG. 24) and ABEs (b of FIG. 24), wherein the red triangles indicate target sequences at which the editing efficiency of one base editor is at least 30% higher than that of the other base editor;

FIG. 25 shows comparison of base editing efficiencies induced by different CGBEs, wherein the red triangles indicate target sequences at which the editing efficiency of one base editor is at least 30% higher than that of the other base editor;

FIG. 26 shows average Cas9 variant-induced indel frequencies at target sequences containing each 4-nt PAM sequence;

FIG. 27 shows comparison of Cas9 variants with different PAM compatibilities (maximum average indel frequencies generated by any one of ten Cas9 variants (the left heat maps) and the corresponding Cas9 variants that show the highest average activities (the right heat maps) at target sequences containing each 4-nt PAM sequence are shown, and when the maximum average indel frequencies are lower than 5% (a of FIG. 27) and 20% (b of FIG. 27), the corresponding candidate PAMs are indicated in white);

FIG. 28 shows correlation of indel frequencies induced by Cas9 variants at targets with four example PAMs;

FIG. 29 shows effects of two-base consecutive transversion mismatches on base editing efficiencies (indel frequencies induced by SpCas9 were analyzed with the same target sequences used to determine base editing efficiencies induced by SpCas9-YE1-BE4max (a of FIG. 29) and SpCas9-ABE8e(V106W) (c of FIG. 29) and are shown as controls);

FIG. 30 shows effects of three-base consecutive transversion mismatches on base editing efficiencies (indel frequencies induced by SpCas9 were analyzed with the same target sequences used to determine base editing efficiencies induced by SpCas9-YE1-BE4max (a of FIG. 30) and SpCas9-ABE8e(V106W) (c of FIG. 30) and are shown as controls);

FIG. 31 shows correlations between predicted DeepNG-BE scores and measured base editing efficiencies (a of FIG. 31) and proportions (b of FIG. 31); and

FIG. 32 shows an architecture of DeepBE, wherein prediction scores of DeepCas9 variants are concatenated with data obtained from base editor variants containing the corresponding Cas9 variants.

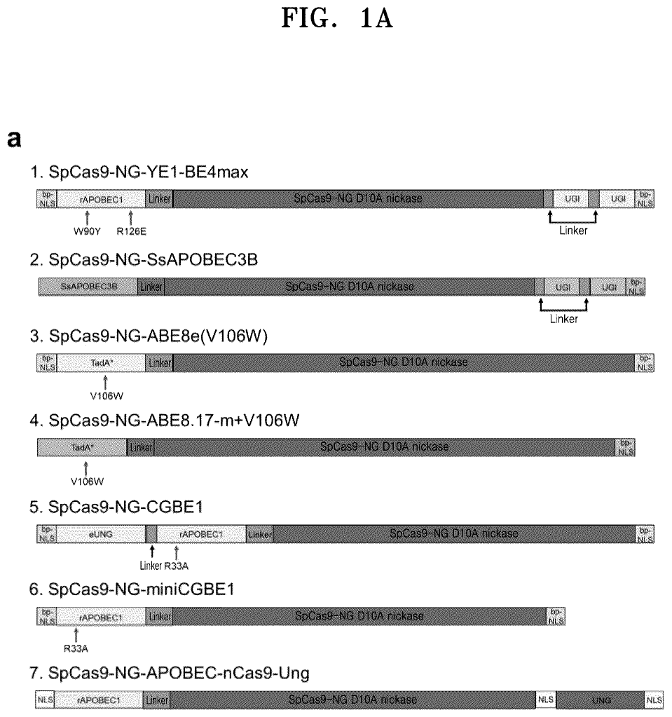

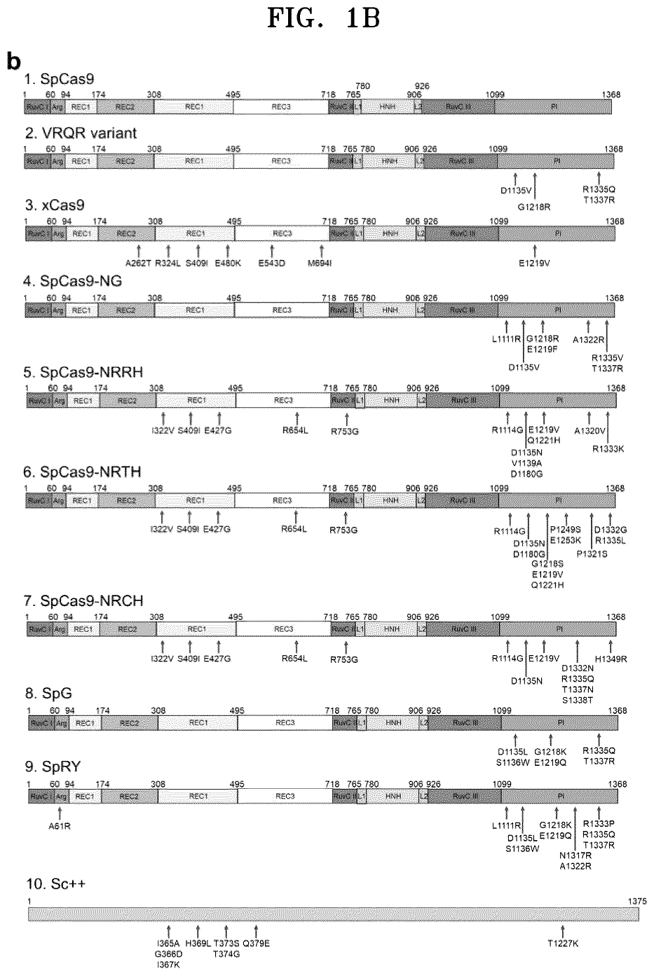

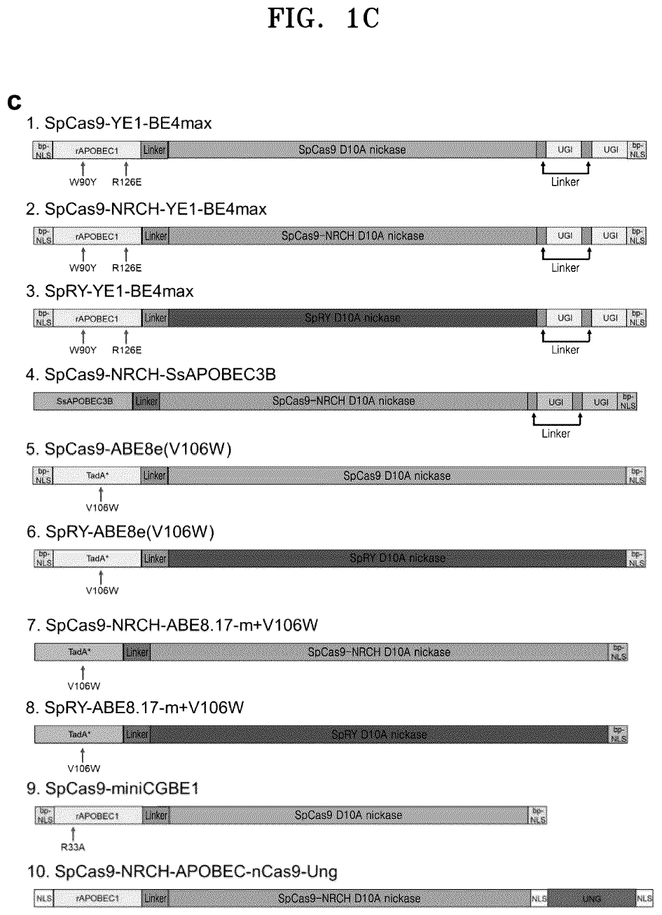

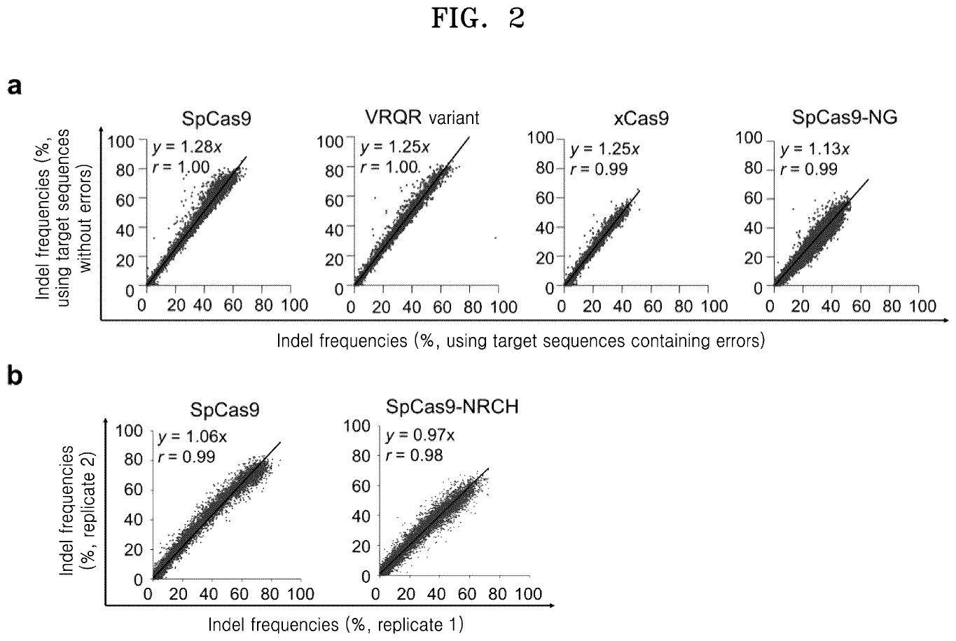

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The disclosure will be described in more detail with reference to the following embodiments. However, the embodiments are for illustrative purposes only and the scope of the disclosure is not limited thereto.

### Embodiment 1. Preparation of Materials

### Embodiment 1-1. Plasmid Construction

To generate the backbone plasmid, the lentiCas9-Blast plasmid (Addgene, 52962) was first digested with XbaI and BamHI restriction enzymes (New England Biolabs, Ipswich, MA), and then treated with 1 μl of quick calf intestinal alkaline phosphatase (New England Biolabs) for 30 min at 37° C. The linearized fragment was then gel purified with a MEGAquick-spin Total Fragment DNA Purification Kit (iNtRON Biotechnology, Seongnam, Republic of Korea) according to the manufacturer's protocol.

PCRs were performed with primers containing desired mutations and Phusion High-fidelity DNA polymerase (New England Biolabs). To attain high protein expression levels, we chose the codons at mutation sites by following GenScript's suggestions in the case of Cas9 variants recognizing different PAMs (i.e., Cas9 PAM variants). As the codons for the deamination domains of base editors, previously used codons from the initial studies were adopted.

The resulting amplicons were gel purified and cloned into the digested lentiCas9-Blast plasmid using NEBuilder Hifi DNA Assembly Master Mix (New England Biolabs); the reaction was allowed to proceed for 1 h at 50° C. For the VRQR variant, xCas9, and SpCas9-NG, we used plasmids that were described in our previous study; these plasmids are available at Addgene (Addgene, 138562, 138565, and 138566).

### Embodiment 1-2. Library Design and Preparation

Library C included 11,994 pairs of guide RNA-encoding sequences and their corresponding target sequences and was used to evaluate the activities of base editors containing Cas9 that recognizes NGG and non-NGG PAMs. This library contained 179 or 180 guide RNA-target pairs for each NNN PAM and 515 previously evaluated endogenous target sequences with 36 different PAMs with five distinct barcodes.

Oligonucleotides for library C were synthesized by Twist Bioscience (San Francisco, CA), PCR-amplified using Phusion High-fidelity DNA polymerase (New England Biolabs), gel-purified, and assembled into the BsmBI (Enzynomics, Daejeon, Republic of Korea)-digested Lenti-gRNA-Puro vector (Addgene 84752) utilizing NEBuilder Hifi DNA Assembly Master Mix (New England Biolabs). After PCR purification using a MEGAquick-spin Total Fragment DNA Purification Kit (iNtRON Biotechnology), the product was transformed into Endura electrocompetent cells (Lucigen, Middleton, WI) to construct the first plasmid library. This plasmid library was then digested with BsmBI restriction enzyme (Enzynomics), treated with quick calf intestinal alkaline phosphatase (New England Biolabs), ligated with an optimized sgRNA scaffold, and transformed into Endura electrocompetent cells (Lucigen). Plasm ids were extracted using a Plasmid Maxi Kit (Qiagen, Hilden, Germany).

### Embodiment 1-3. Cell Culture and Production of Lentivirus

HEK293T cells (American Type Culture Collection) were maintained in Dulbecco's modified Eagle's Medium (DMEM; Gibco, Waltham, MA) that was supplemented with 10% fetal bovine serum (FBS; Gibco). HEK293T cells were seeded the day before transfection and treated with chloroquine diphosphate for up to 5 h on the day of transfection. Opti-MEM reduced-serum medium (Gibco) was mixed with 120 μl of Polyethylenimine reagent, 20 μg of lentiviral vector, 15 μg of PAX2, and 5 μg of pMD2.G for a final volume of 1 ml, after which the solution was incubated at room temperature for 15-20 min and then added to the cell culture medium. The next day, the lentivirus-containing medium was removed and replaced with fresh DMEM (Gibco) supplemented with 10% FBS (Gibco). After 48 h of transfection, we directly harvested the variant virus-containing supernatant or added Benzonase (Enzynomics) and Benzonase buffer to remove the residual library plasm ids for the lentiviral plasmid library before harvesting the supernatant. The harvested supernatant was then stored at −80° C.

### Embodiment 1-4. Generation of Stable Cell Lines and Transduction of the Lentiviral Plasmid Library

For lentiviral variant-expressing cell lines, cells that had been infected at 0.15 MOI were chosen for further evaluation and continuously maintained with 20 μg of Blasticidin S (InvivoGen, San Diego, CA). The variant-expressing cells were seeded a day before lentiviral plasmid library transduction and then infected at an MOI of 0.4 in the presence of 10 μg ml-1 of polybrene. After 18-19 h of transduction, the medium was exchanged for fresh medium supplemented with 2 μg ml-1 of puromycin (Invitrogen, Waltham, MA) and 20 μg ml-1 of Blasticidin S (InvivoGen). A summary of the stable cell lines and the number of cells that we utilized for each library is provided below.

(1) Library A (8×10⁷ cells per cell line; 2×10⁷ cells were seeded into four 15-cm dishes)

i. Cas9 variants (harvested at Day 4)

SpCas9, VRQR variant, xCas9, SpCas9-NG, SpCas9-NRRH, SpCas9-NRTH, SpCas9-NRCH, SpG, SpRY, and Sc++.

ii. Base editor variants based on SpCas9 (harvested at Day 10)

YE1-BE4max and ABE8e(V106W).

(2) Library B (2×10⁸ cells per cell line; 2.5×10⁷ cells were seeded into eight 15-cm dishes)

i. Cas9 variants (harvested at Day 4)

SpCas9, VRQR variant, xCas9, SpCas9-NG, SpCas9-NRRH, SpCas9-NRTH, SpCas9-NRCH, SpG, SpRY, and Sc++.

ii. Base editor variants based on SpCas9 (harvested at Day 6)

YE1-BE4max and ABE8e(V106W).

iii. CBE variants based on SpCas9-NG (harvested at Day 6)

YE1-BE4max and SsAPOBEC3B.

iv. ABE variants based on SpCas9-NG (harvested at Day 6)

ABE8e(V106W) and ABE8.17m-V106W.

v. C-to-G base editor variants based on SpCas9-NG (harvested at Day 6)

CGBE1, miniCGBE1, and APOBEC-Cas9n-Ung.

(3) Library C (8×10⁷ cells per cell line; 2×10⁷ cells were seeded into four 15-cm dishes)

i. CBE variants (harvested at Day 6)

SpCas9-NRCH-YE1-BE4max, SpRY-YE1-BE4max, and SpCas9-NRCH-SsAPOBEC3B.

ii. ABE variants (harvested at Day 6)

SpRY-ABE8e(V106W), SpCas9-NRCH-ABE8.17m-V106W, and SpRY-ABE8.17m-V106W.

iii. CGBE variants (harvested at Day 6)

SpCas9-miniCGBE1 and SpCas9-NRCH-APOBEC-Cas9n-Ung.

### Embodiment 2. Experimental Method and Outcome Measurement

### Embodiment 2-1. Deep Sequencing

Genomic DNA was isolated using a Wizard Genomic DNA Purification Kit (Promega, Fitchburg, WI) according to the manufacturer's instructions. Integrated sequences including the sgRNA-encoding sequence, barcode, and target sequence were PCR amplified with 2×Taq PCR Smart Mix (Solgent) from 48 separate 50-μl reactions with 5 μg of genomic DNA (Library A and C; a total of 240 μg of genomic DNA per technical replicate) or 96 separate 50-μl reactions with 10 μg of genomic DNA (Library B; a total of 480 μg of genomic DNA per technical replicate). After pooling, PCR products were purified using a MEGAquick-spin Total Fragment DNA Purification Kit (iNtRON Biotechnology) according to the manufacturer's protocol. The amplicons were then sequenced on a NovaSeq 6000 System (Illumina) or a Nextseq 2000 System (Illumina).

### Embodiment 2-2. Evaluation of Nuclease and Base Editor Variants Activities

After deep sequencing, the data were analyzed using in-house Python scripts (see code availability). To improve the accuracy of data, we eliminated pairs that contained i) errors within guide RNAs, scaffolds, or barcodes, which were generated during the process of oligo synthesis, PCR amplifications, or sequencing or ii) shuffling between barcodes and guide RNA sequences.

for analysis of the activities of Cas9 variants, we filtered out the data that had fewer than 100 (Library A) or 200 (Library B) total read counts and had background indel frequencies greater than 8%. For analysis of intended base conversions by the base editor variants, we excluded the data with fewer than 100 total read counts. For analysis of total base editing, we discarded the sequences with less than 100 total read counts and background base editing efficiencies greater than 8%.

$$[ Indel frequencies and total base editing ( \% ) ] = \frac{\begin{matrix}Indel read counts - ( Total read counts \times \\ background indel frequency ) / 100\end{matrix}}{\begin{matrix}Total read counts - ( Total read counts \times \\ background indel frequences ) / 100\end{matrix}} \times 100$$

$$[ Base editor efficiencies at each position ( intended base conversions ; \% ) ] = \frac{\begin{matrix}Total read counts of intended target nucleotide \\ conversions at each position\end{matrix}}{Total read counts} \times 100$$

$$[ Base editor outcome proportion ] = \frac{\begin{matrix}Total read counts of a unique \\ base - edited outcome sequence\end{matrix}}{\begin{matrix}Total read counds of converted \\ sequences within the wide windows\end{matrix}}$$

$${X}_{normalized} = \frac{( x - {x}_{min} )}{( {x}_{max} - {x}_{min} )}$$

### Embodiment 2-3. Western Blot Analysis

Harvested cells were lysed in a buffer containing 20 mM HEPES, 150 mM NaCl, 1% NP-40, 0.25% sodium deoxycholate, and 10% glycerol to which a 1:100 dilution of protease inhibitor cocktail (Cell Signaling Technology) had been added. The mixture was incubated on ice for 20 min. The resulting cell lysate solutions were centrifuged at 13,000 g for 15 min at 4° C. A Bradford Protein Assay Kit (Pierce) was used to determine the total protein concentration in the supernatant. Proteins (30 μg per well) were separated on 4-12% Bis-Tris gels, which were run in 1× NuPAGE MOPS SDS running buffer (Invitrogen) at 120 V for 2 h. Next, an XCell II Blot Module (Invitrogen) was used to transfer the proteins onto a 0.45-μm Invitrolon polyvinylidene difluoride (Invitrogen) membrane; transfer took place in 10% (vol/vol) methanol in 1× NuPAGE Transfer Buffer on ice. After blocking with 5% BSA for 1 h, the membranes were then probed with primary antibodies recognizing SpCas9 (cat. no. 844301, BioLegend) and β-actin (cat. no. sc-47778, Santa Cruz Biotechnology) diluted 1:1,000 and 1:2,000, respectively, in 5% BSA overnight at 4° C. The membranes were then washed and incubated for 1 h at room temperature with horseradish peroxidase-conjugated goat anti-mouse IgG secondary antibodies (cat. no. sc-516102, Santa Cruz Biotechnology) at 1:3,000 dilution. The antibodies were visualized with West-Q Pico ECL Solution (GenDEPOT) using the ImageQuant LAS-4000 digital imaging system (GE Healthcare).

### Embodiment 2-4. Deep Learning Models

We randomly split our data into training and test data sets, and 5-fold cross-validation was performed for training. For prediction of indel frequencies generated by the Cas9 variants, 26,960-27,342 and 1,003-3,529 target sequences from library A and B were utilized for training and test data sets, respectively. In library B, we used 12,553-16,624 and 8,507-86,822 target sequences for efficiency and pattern training, respectively, for base editor variants based on SpCas9-NG. From library C, 2,378-8,287 target sequences were used for efficiency training for base editor variants containing Cas9 variants.

Input sequences were converted into numerical representations by one-hot encoding, and zero-padding was applied for maintaining the number of input sequences. Input sequence features were extracted with one convolution layer consisting of 1,000 or 2,000 filters of 10-nt length for DeepCas9variants, 1,024 filters of 3-nt length for efficiency models of DeepNG-BE, and 256 or 1,024 filters of 3-nt length for proportion models of DeepNG-BE. As with the deep reinforcement learning algorithm, we omitted the pooling layers to maintain local information, as previously described. To create one-dimensional input, the Flatten layer was used, and every model consisted of two or three dense layers. In the first or second layers, 1000 or 1500 nodes for DeepCas9variants, 1500 or 2000 nodes for efficiency models of DeepNG-BE, and 2500 or 5000 nodes for proportion models of DeepNG-BE were adopted. For the last dense layer, 100 nodes for DeepCas9variants and efficiency models of DeepNG-BE and 31, 127, 255, and 31 nodes for proportion models of YE1-BE4max, SsAPOBEC3B, ABEs, and CGBEs, respectively, were utilized. The output layer of DeepCas9variants generated prediction scores of the Cas9 variants, and the prediction scores of DeepNG-BE were generated by multiplying the outputs of the efficiency and proportion models of DeepNG-BE.

Because base editor outcomes are determined by deaminases, proportion models of DeepNG-BE were adopted. For developing efficiency models, data obtained using base editors containing SpCas9-NG or Cas9 variants were utilized to generate 7, 9, or 10-nt input sequences. The input sequences were converted into a binary matrix by one-hot encoding, and zero-padding was used. In the convolution layer, 256, 512 or 1,024 nodes were adopted, and the extracted features were flattened. To consider the guide sequence preferences of Cas9 variants, the DeepCas9variants prediction scores were concatenated in the Flatten layer. The output layers of the efficiency and proportion models were multiplied to generate the prediction scores for the base editors containing Cas9 variants.

Dropout layers were utilized to avoid overfitting with a rate of 0.3, and a rectified linear unit (ReLU) was used as the activation function for every layer. The outputs of DeepCas9variants and the efficiency models of DeepNG-BE and DeepBE were linearly transformed. For the output layer of the proportion models of DeepNG-BE and DeepBE, a softmax function was applied as an activation function. The mean absolute error was adopted as the loss function, and an Adam optimizer with a learning rate of 10-4 was used. TensorFlow was utilized for developing our models.

### Embodiment 2-5. Statistical Significance

The Wilcoxon rank-sum test was used in FIG. 19g,h. Statistical significance was analyzed through SPSS Statistics (version 25, IBM).

### Experimental Example 1. High-Throughput Evaluations of the Activities of Cas9 and Base Editor Variants

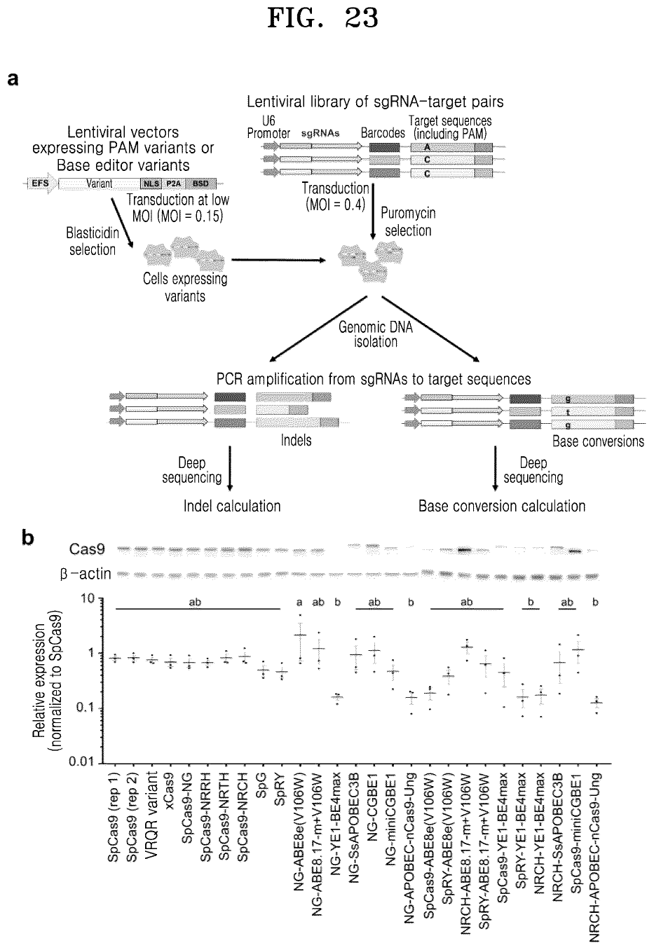

To compare the base editing and nuclease activities of variants, we first generated cell lines expressing these variants at comparable levels. Given that codon usage affects protein expression levels, we used the same codons present in the widely used SpCas9-encoding sequence for the Cas9 variants, except at the mutation sites, where codons were selected based on suggestions from GenScript that resulted in high expression levels of SpCas9 base editors. HEK293T cells were transduced with individual lentiviral vectors encoding Cas9 or base editor variants at a multiplicity of infection (MOI) of 0.15, so that every transduced cell had only one copy of the Cas9 or base editor variant-encoding sequence; untransduced cells were removed by blastidicin S selection. Western blotting showed that the levels of most Cas9 and base editor variant proteins were comparable except that NG-ABE8e(V106W) showed statistically significant higher protein levels than three YE1-BE4max variants (NG-YE1-BE4max, SpRY-YE1-BE4max, NRCH-YE1-BE4max) and two APOBEC-nCas9-Ung variants (NG-APOBEC-nCas9-Ung, NRCH-APOBEC-nCas9-Ung) (FIG. 23b).

To measure the activities of base editors and Cas9 nucleases at a large number of target sequences, we used a high-throughput approach involving pairwise libraries of sgRNA-encoding and target sequences as we previously did to evaluate Cas9 and base editor activities. We used previously prepared libraries A and B, as well as a library generated in the current study named library C, which included 11,802, 23,679, and 11,994 pairs of sgRNA-encoding and target sequences, respectively. Library A contained 8,130 pairs for the evaluation of PAM compatibilities, and 2,940 and 732 pairs for assessing mismatch tolerance with NGG and non-NGG PAM sequences, respectively. Library B included 8,744, 12,093, and 2,660 pairs with NGG, NGH, and non-NG PAM sequences, respectively, to measure the activities of Cas9 and base editor variants with diverse PAM compatibilities at large numbers of target sequences. Library C had 179 or 180 pairs for each NNN PAM sequence to determine the activities of base editors containing versions of Cas9 that recognize NGG and non-NGG PAMs.

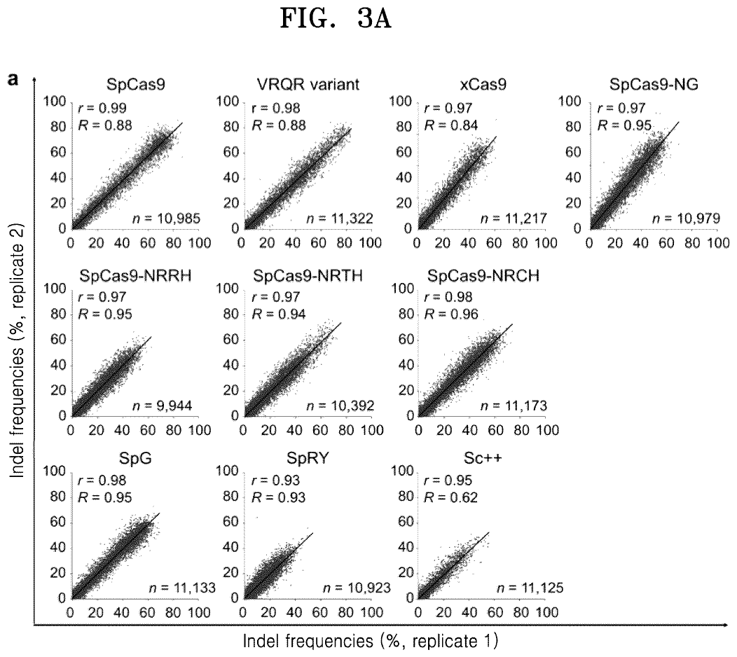

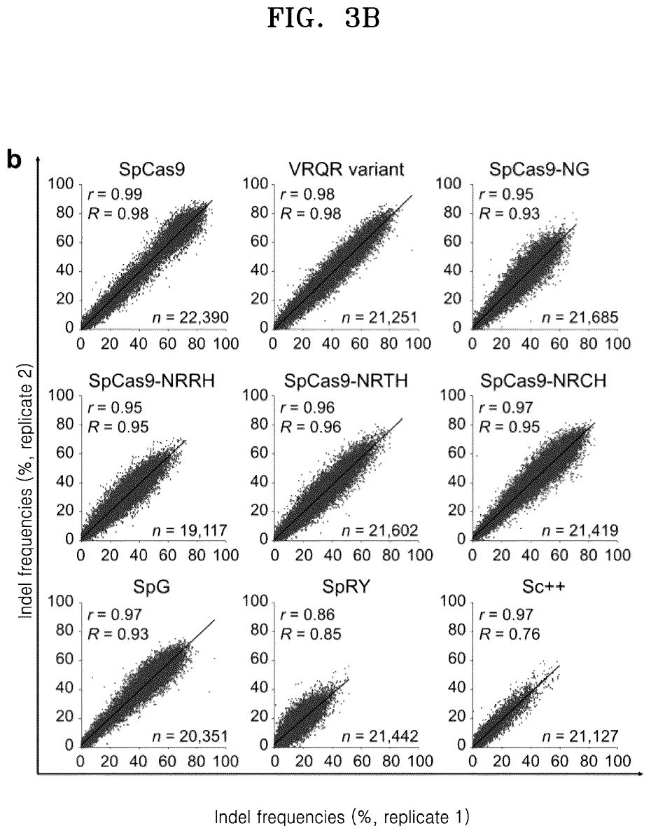

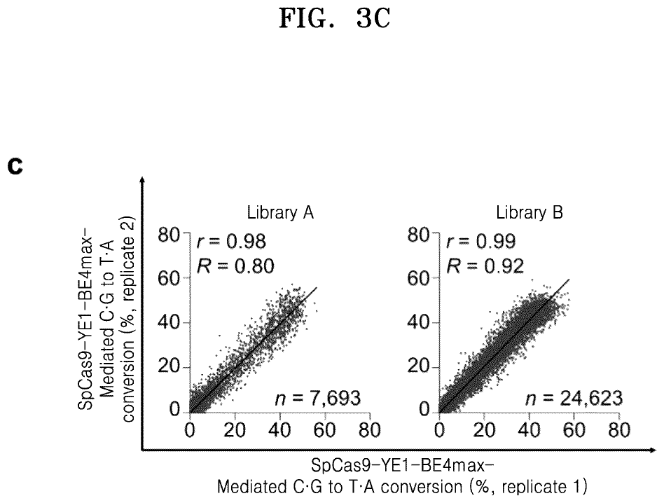

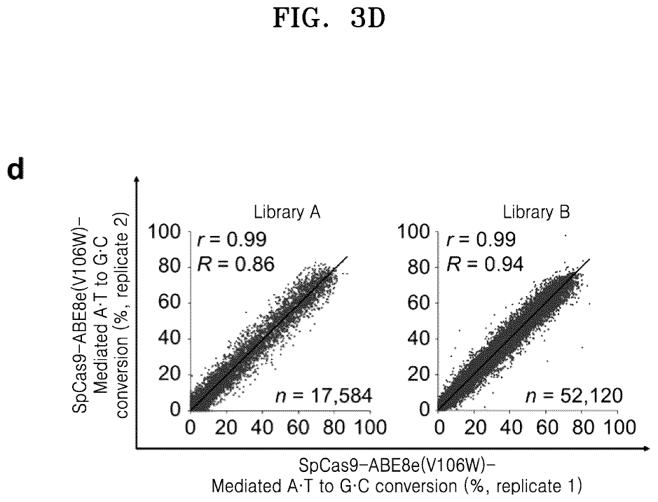

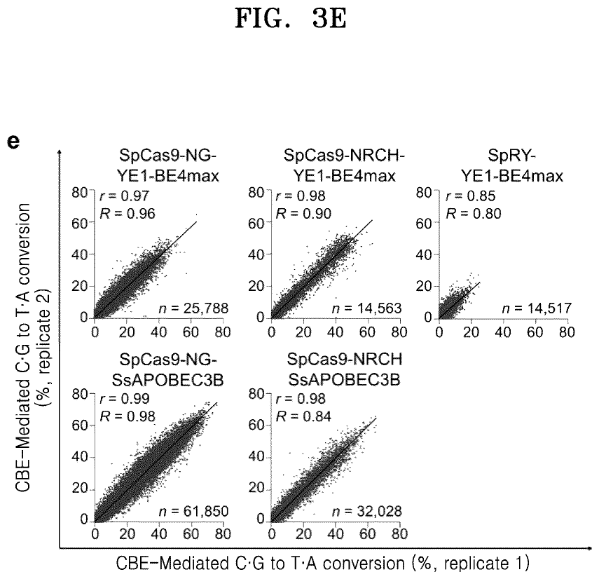

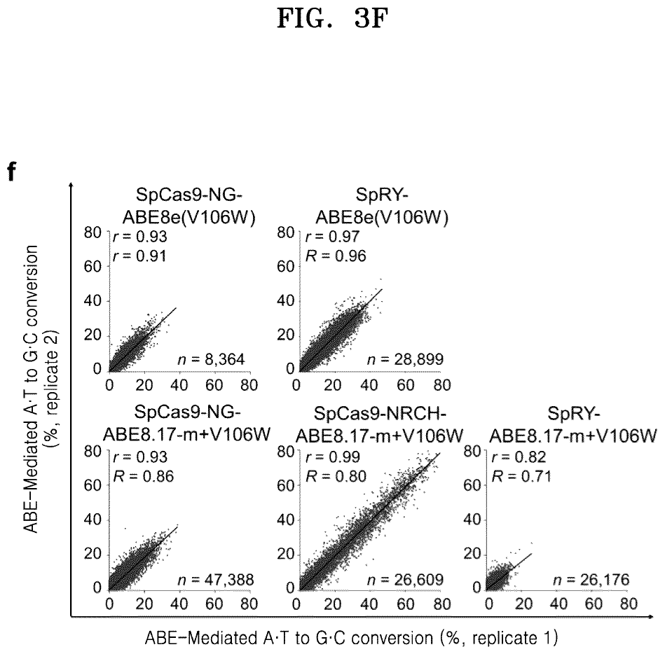

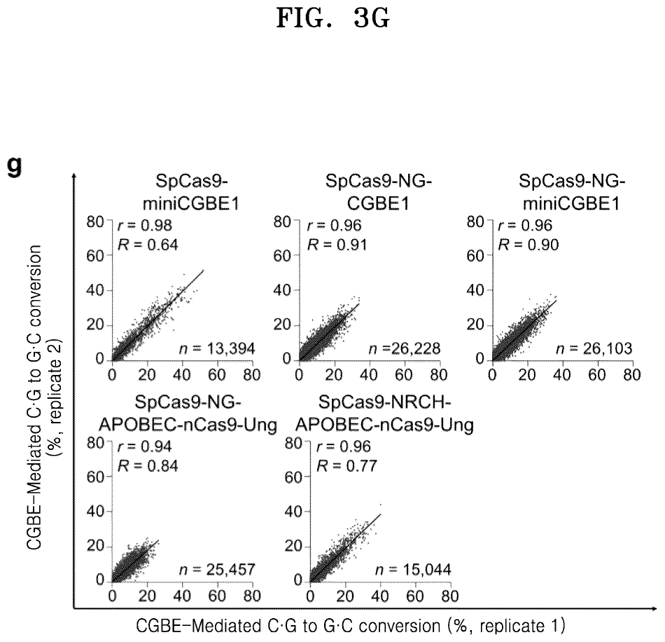

To improve the accuracy of data, we i) eliminated sequences that contained technical errors within sgRNAs, scaffolds, or barcodes, which were generated during the process of oligo synthesis, PCR amplifications, or sequencing and ii) removed sequences in which shuffling had occurred in the barcode or sgRNA regions during lentivirus production. We previously showed that such errors and shuffling can, albeit slightly, lead to an underestimation of the editing efficiencies. When we compared indel frequencies before and after removing sequencing reads that contained errors or shuffled sequences, target sequences without errors or shuffling had higher indel frequencies than those with errors or shuffling and we observed high correlations between these two values as expected (FIG. 2a). Because the indel frequencies were highly correlated between two biological replicates (FIG. 2b) and technical replicates (FIG. 3), we combined the results from two replicates to draw more generalized and accurate conclusions.

### Experimental Example 2. The Editing Window, Product Purity, and Preferred Motifs for CBE and ABE Variants

Deaminases are an essential component of base editors and applications of base editing have often been limited due to insufficient editing activities or DNA and RNA off-target effects, especially those that are Cas9-independent. CBEs and ABEs with advanced base-converting domains, which include the CBEs YE1-BE4max and SsAPOBEC3B and the ABEs ABE8e(V106W) and ABE8.17-m+v106W, have been reported to have high on-target activity and minimal off-target effects. However, the activities of these base editors have not been extensively compared at a large number of target sequences, making the selection of the most appropriate base editor version difficult. Thus, to comparatively evaluate the activities, editing windows, and specificities of base editors containing these advanced base-converting domains, we combined the base-converting domains with SpCas9-NG, the SpCas9 variant with broad PAM compatibilities.

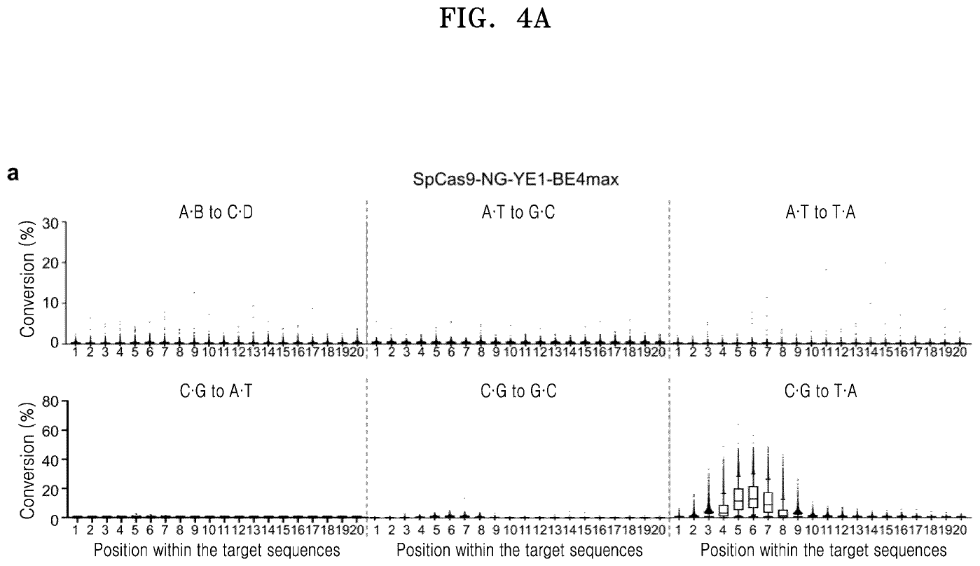

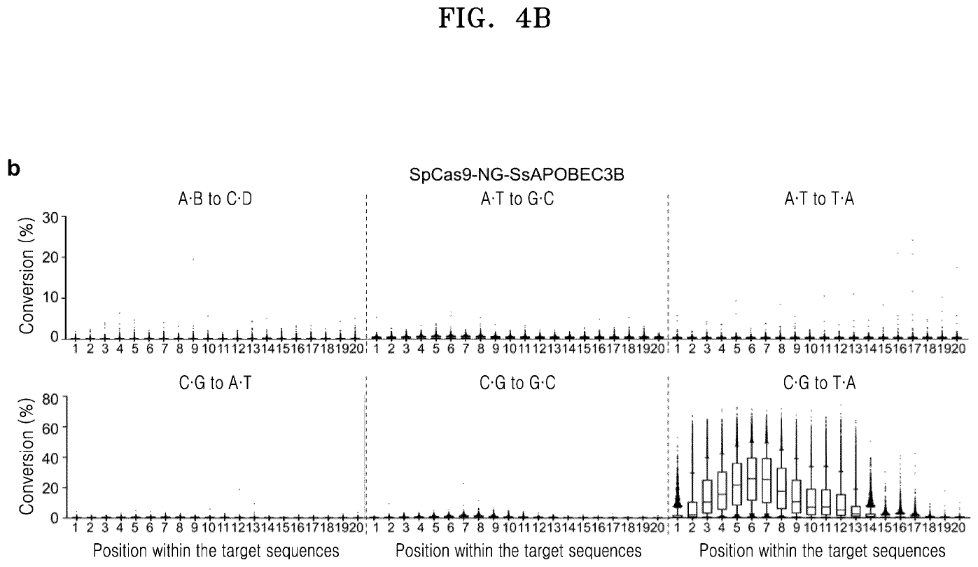

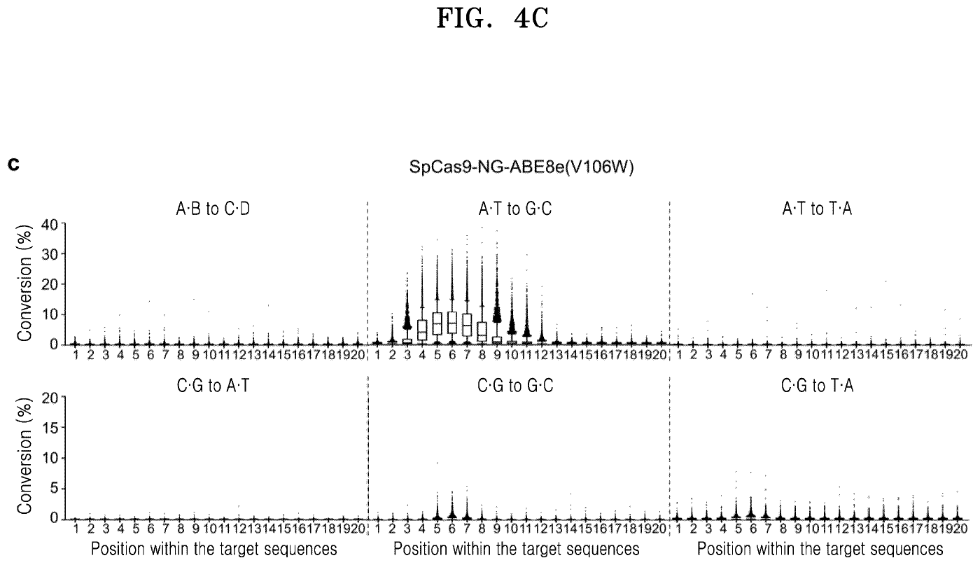

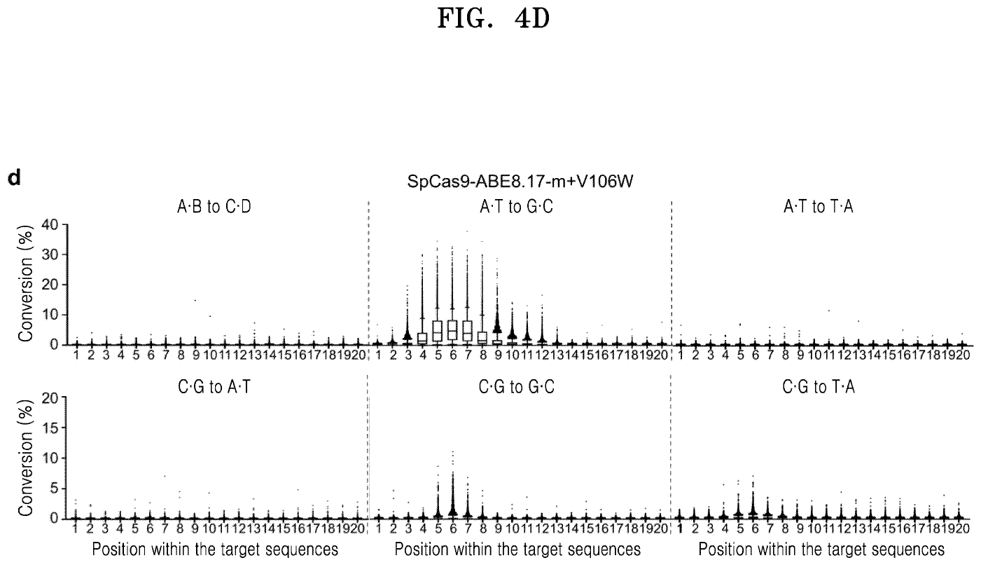

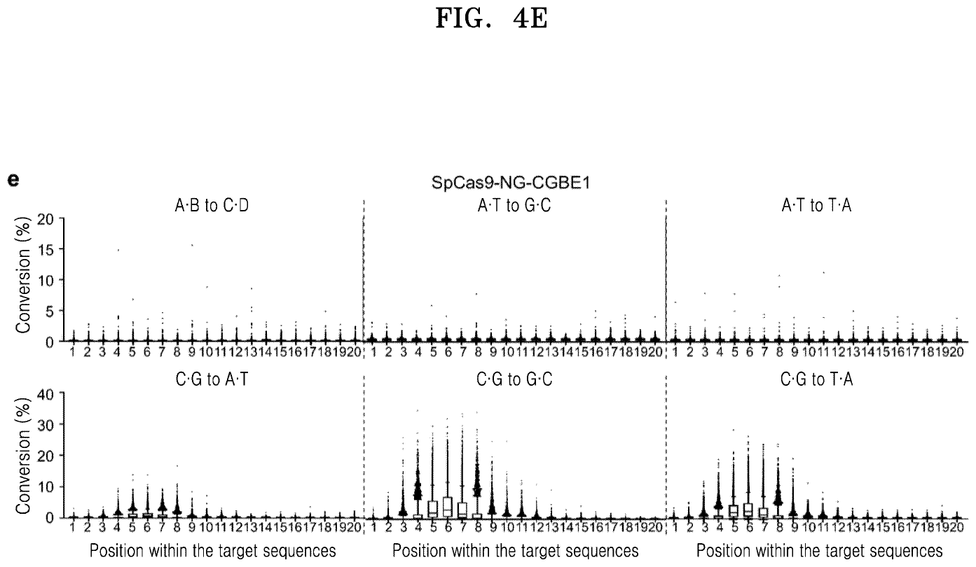

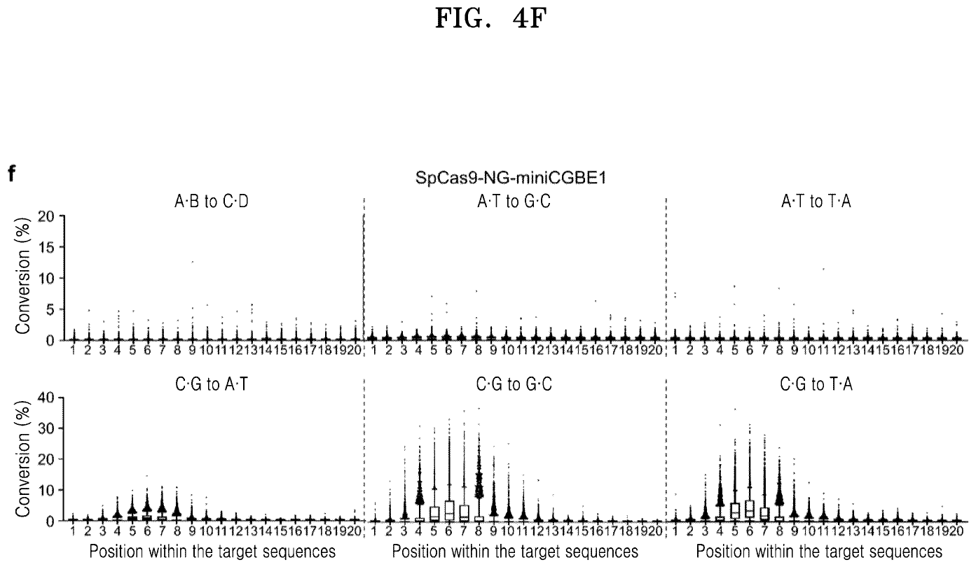

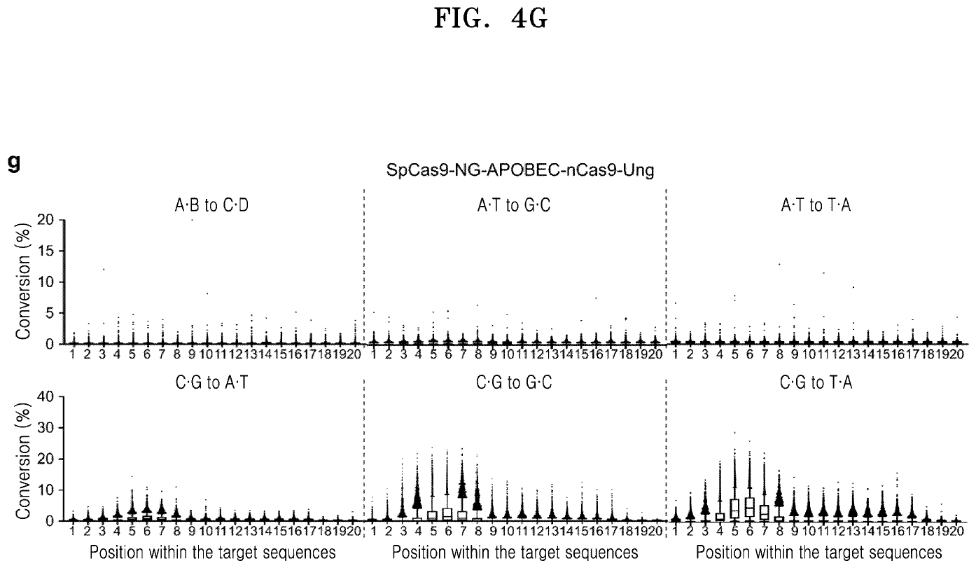

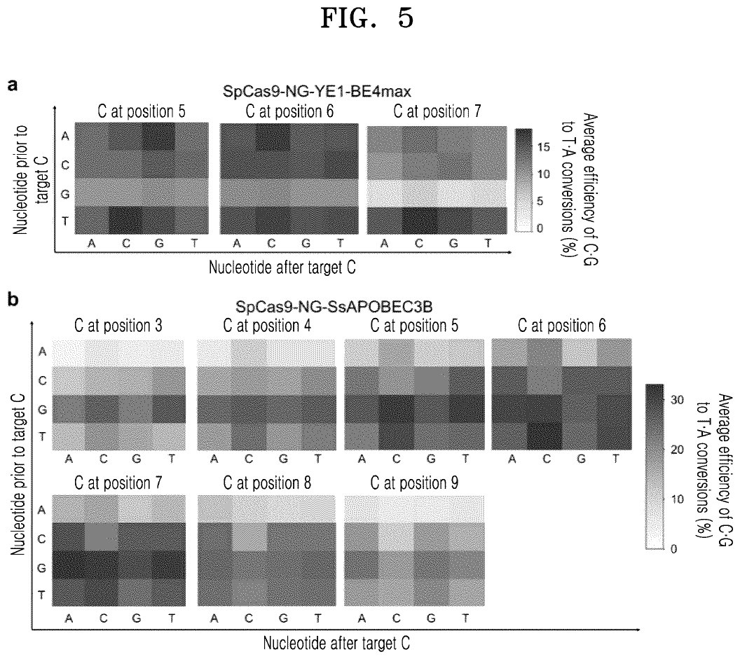

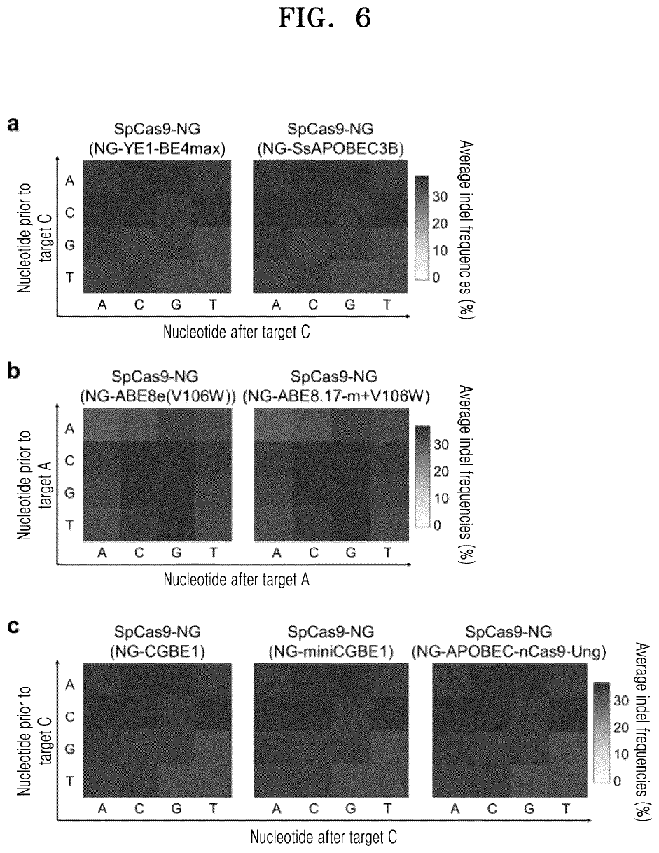

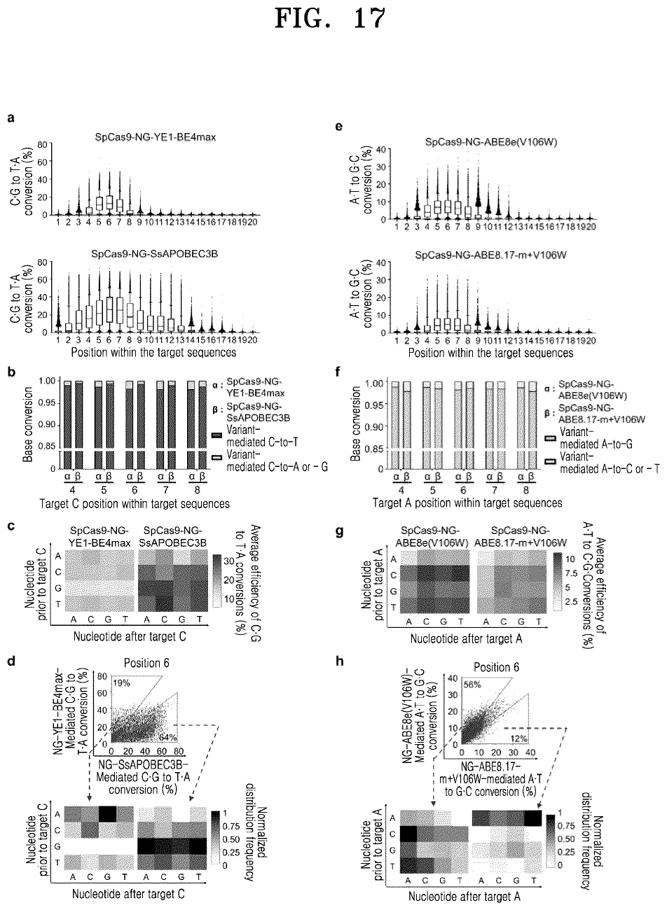

We first determined the windows for the intended base conversions. Although the base editing activities of both SpCas9-NG-YE1-BE4max and SpCas9-NG-SsAPOBEC3B peaked at position 6 (numbered such that position 20 of the guide sequence is immediately adjacent to the NGG PAM and position 1 is 20 base pairs away from the PAM), the editing window of SpCas9-NG-SsAPOBEC3B spans positions 2 to 13, which is broader than that of SpCas9-NG-YE1-BE4max, which spans positions 4 to 8 (FIG. 17a). The two CBEs were only rarely observed to induce base conversions other than C•G to T•A (FIG. 4a,b). The purity of SpCas9-NG-YE1-BE4max and SpCas9-SsAPOBEC3B-induced C to T versus C to A or G conversions ranged from 98.1% to 98.9% and from 98.7% to 99.5%, respectively, depending on the position (FIG. 17b). When we analyzed which motifs were preferred by the two CBEs, we found that the motifs differed; SpCas9-NG-YE1-BE4max preferred (A/C/T)cN (with c being the target nucleotide) motifs, whereas SpCas9-NG-SsAPOBEC3B showed high activities at TcC and Gc(A/C/T) motifs (FIG. 17c). Similar motif effects were observed for other positions within the base editing window (FIG. 5). These effects were not observed in the generation of indels by the corresponding SpCas9-NG (FIG. 6), supporting that such effects are attributable to the base-converting domains.

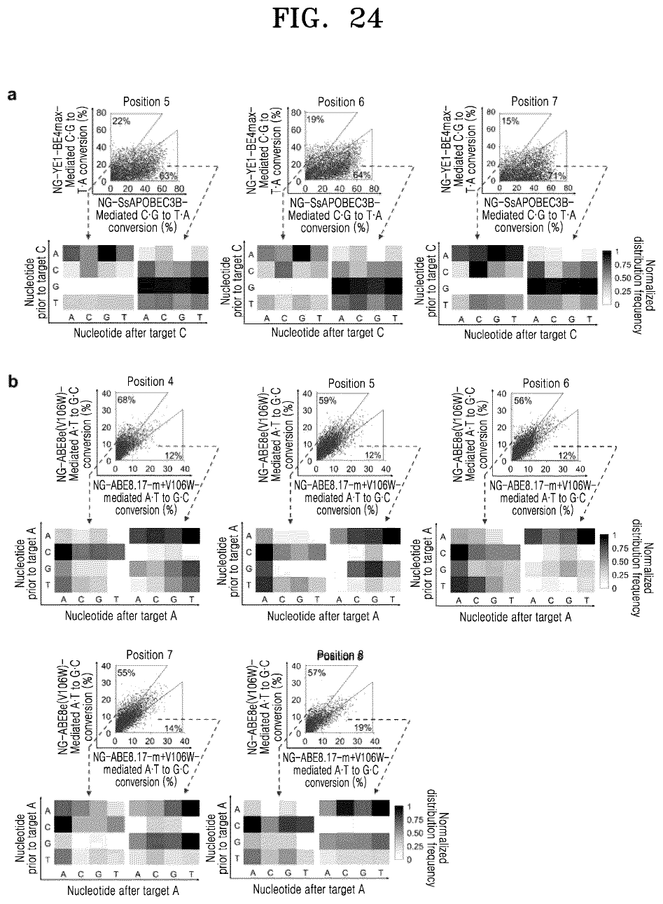

The overall base editing activities of SpCas9-NG-SsAPOBEC3B were higher than those of SpCas9-NG-YE1-BE4max; the median editing activities of SpCas9-NG-SsAPOBEC3B and SpCas9-NG-YE1-BE4max were 26% and 13%, respectively, at position 6. However, at some target sequences, the base editing activities of SpCas9-NG-YE1-BE4max were higher than those of SpCas9-NG-SsAPOBEC3B. When analyzed at position 6, the base editing activities of SpCas9-NG-YE1-BE4max and SpCas9-NG-SsAPOBEC3B were at least 30% higher than those of SpCas9-NG-SsAPOBEC3B and SpCas9-NG-YE1-BE4max at 19% and 64% of the target sequences, respectively (FIG. 17d). The 19% of the target sequences at which SpCas9-NG-YE1-BE4max showed higher activities than SpCas9-NG-SsAPOBEC3B were strongly enriched with AcG motifs and slightly enriched with (A/C/T)cN motifs. Conversely, the 64% of the target sequences at which SpCas9-NG-SsAPOBEC3B showed higher activities than SpCas9-NG-YE1-BE4max were strongly enriched with GcN motifs and depleted for AcG motifs. Similar motif effects were found at the other positions within the base editing window (FIG. 24a). Thus, one can choose a preferred base editor with an appropriate base-converting domain depending on the motif surrounding the target nucleotide c for efficient base editing.

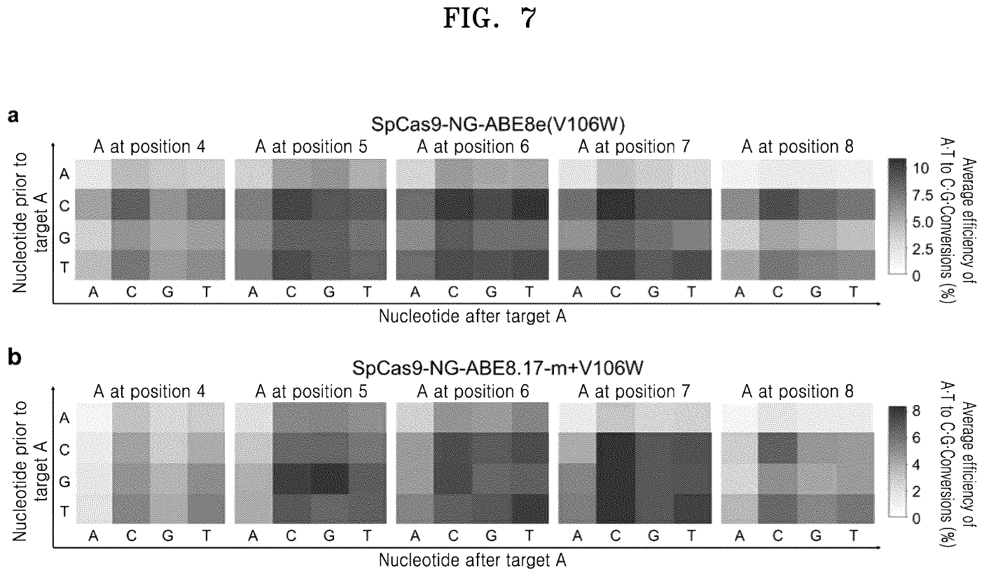

The editing windows of SpCas9-NG-ABE8e(V106W) and SpCas9-NG-ABE8.17-m+V106W were similar; both spanned positions 4 to 8, with activity peaking at position 6 (FIG. 17e). The two ABEs were only rarely observed to induce base conversions other than A•T to G•C, with the exception of low levels of C•G to G•C and C•G to T•A editing, which peaked at position 6 (FIG. 4c,d). The purity of SpCas9-NG-ABE8e(V106W) and SpCas9-NG-ABE8.17-m+V106W-induced A to G versus A to C or G conversions ranged from 98.1% to 98.8% and 97.8% to 98.5%, respectively, depending on the position (FIG. 17f). When we analyzed which motifs were preferred by the two ABEs, we found that the preferences were similar: Ca(C/T) and TaB motifs (FIG. 17g). Similar motif effects were found for other positions within the base editing window (FIG. 7), as similarly observed in experiments with CBEs described above.

The overall base editing activities of SpCas9-NG-ABE8e(V106W) were slightly higher than those of SpCas9-NG-ABE8.17-m+V106W; the median editing activities of SpCas9-NG-ABE8e(V106W) and SpCas9-NG-ABE8.17-m+V106W were 7% and 5%, respectively, at position 6. However, at some target sequences, the base editing activities of SpCas9-NG-ABE8.17-m+V106W were higher than those of SpCas9-NG-ABE8e(V106W). When analyzed at position 6, the base editing activities of SpCas9-NG-ABE8e(V106W) and SpCas9-NG-ABE8.17-m+V106W were at least 30% higher than those of SpCas9-NG-ABE8.17-m+v106W and SpCas9-NG-ABE8e(V106W) at 56% and 12% of the target sequences, respectively. The 56% of the target sequences at which SpCas9-NG-ABE8e(V106W) showed higher activities than SpCas9-NG-ABE8.17-m+V106W were strongly enriched with CaA motifs and slightly enriched with Ca(C/G/T)B and Ta(A/C) motifs, whereas the 12% of the target sequences at which SpCas9-NG-ABE8.17-m+V106W showed higher activities than SpCas9-NG-ABE8e(V106W) were strongly and slightly enriched with AaT and AaV motifs, respectively (FIG. 17h). Similar motif effects were found at the other positions within the base editing window (FIG. 24b). Thus, one can choose a preferred base editor with an appropriate base-converting domain depending on the motif surrounding the target nucleotide A for efficient base editing.

### Experimental Example 3. The Editing Window, Product Purity, and Preferred Motifs for CGBE Variants

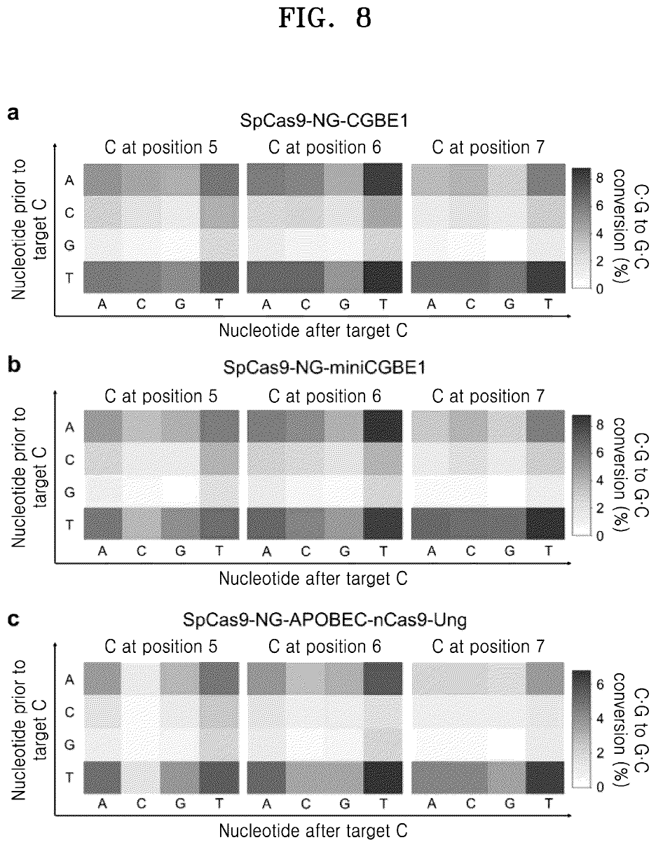

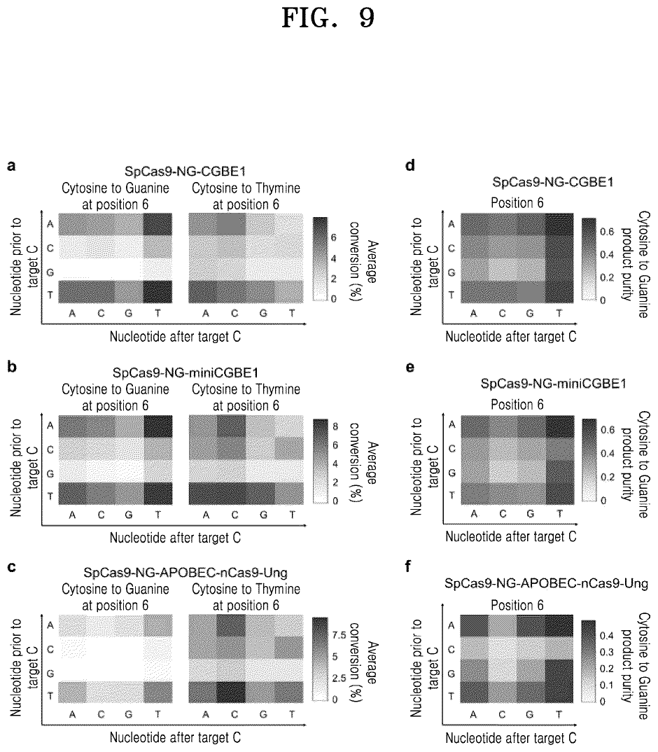

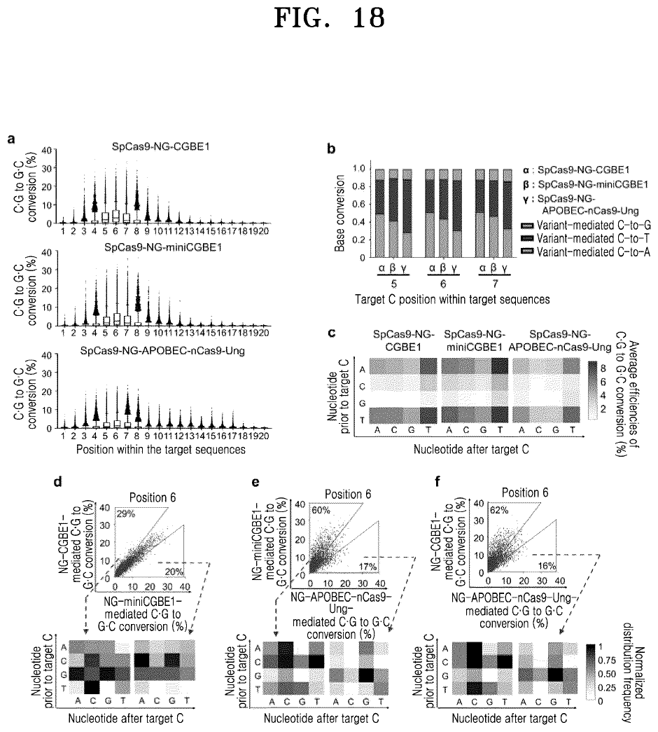

We compared the activities of three CGBE variants based on the SpCas9-NG nickase. The C•G to G•C editing windows of these three variants spanned positions 5 to 7, with activity peaking at position 6 (FIG. 18a). SpCas9-NG-CGBE1, SpCas9-NG-miniCGBE1, and SpCas9-NG-APOBEC-nCas9-Ung exhibited overall C•G to G•C editing activities in the order listed, with median activities of 3.0%, 2.6%, and 1.4% at position 6. In addition to C•G to G•C editing, C•G to T•A and C•G to A•T base conversions were frequently observed for all three CGBE variants (the median C•G to T•A conversion activities of SpCas9-NG-CGBE1, SpCas9-NG-miniCGBE1, and SpCas9-NG-APOBEC-nCas9-Ung were 2.4%, 3.5%, and 4.5% and the median C•G to A•T base conversion efficiencies were 0.7%, 0.8%, and 0.9%, respectively) (FIG. 4e-g); the purity of C•G to G•C editing by SpCas9-NG-CGBE1, SpCas9-NG-miniCGBE1, and SpCas9-NG-APOBEC-nCas9-Ung was only 51%, 44%, and 30%, respectively, at position 6 (FIG. 18b). Similar findings regarding the purity of C•G to G•C editing (the highest for SpCas9-NG-CGBE1 and the lowest for SpCas9-NG-APOBEC-nCas9-Ung) were also observed at positions 5 and 7 (FIG. 18b). When we analyzed the motifs preferred by the CGBEs, we found that their preferences were similar; (A/T)cT was the most preferred and (A/T)c(A/C/G) was the second most preferred at position 6 (FIG. 18c). Similar motif effects were found at positions 5 and 7 within the editing window (FIG. 8), as was likewise observed in experiments with CBEs and ABEs described above. Importantly, the preferred motifs for C•G to G•C versus C•G to T•A editing differed (FIG. 9a-c) and thus, the purity of C•G to G•C editing also dramatically differed depending on the nucleotides adjacent to the target cytosine (FIG. 9d-f). Specifically, a higher purity of C•G to G•C editing was observed at (A/G/T)cT motifs for both SpCas9-NG-miniCGBE1 and SpCas9-NG-APOBEC-nCas9-Ung and at NcT motifs for SpCas9-NG-CGBE1.

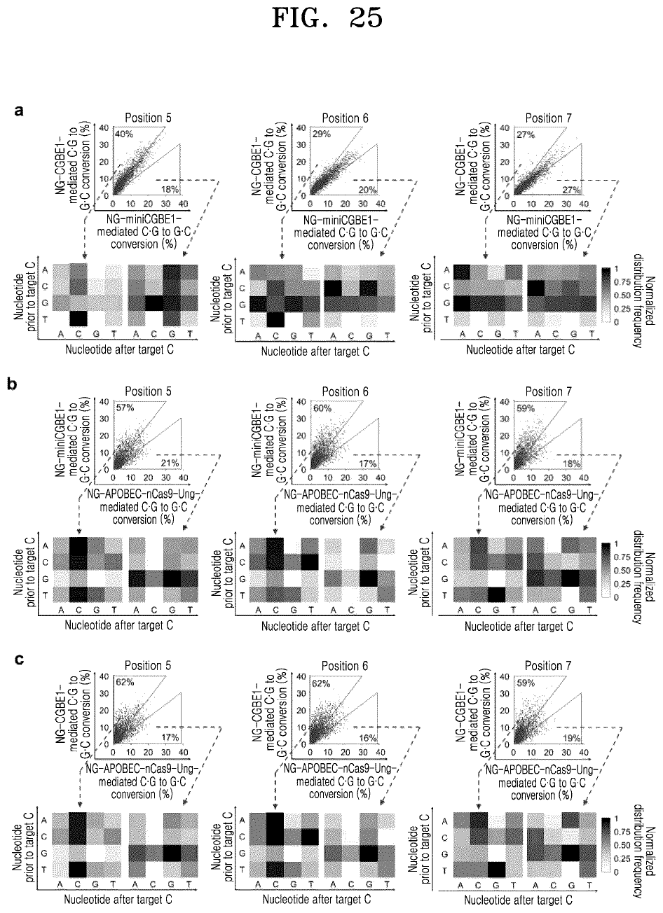

Although the overall C•G to G•C editing activities of SpCas9-NG-CGBE1, SpCas9-NG-miniCGBE1, and SpCas9-NG-APOBEC-nCas9-Ung ranged from higher to lower in the order listed, their relative editing efficiencies differed depending on the target sequence. At some target sequences, the base editing activities of SpCas9-NG-miniCGBE1 and SpCas9-NG-APOBEC-nCas9-Ung were higher than those of SpCas9-NG-CGBE1 and SpCas9-NG-miniCGBE1, respectively. When analyzed at position 6, the C•G to G•C base editing activities of SpCas9-NG-miniCGBE1 were at least 30% higher than those of SpCas9-NG-CGBE1 at 20% of the target sequences and those of SpCas9-NG-APOBEC-nCas9-Ung were also at least 30% higher than those of SpCas9-NG-miniCGBE1 and SpCas9-NG-CGBE1 at 17% and 16% of the target sequences, respectively (FIG. 18d-f). These target sequence-dependent differences in the relative efficiencies of the CGBE variants were associated with differences in preferred motifs between the variants. Similar motif effects were found for the other positions within the base editing window (FIG. 25). Thus, as for CBEs and ABEs, a preferred CGBE variant can be chosen depending on the motif surrounding the target nucleotide C for efficient base editing.

### Experimental Example 4. Determination of Cas9 Variants that have the Highest Activity at a Given PAM Sequence

We previously found that that, among the variants we tested, SpCas9-NG has the broadest PAM compatibilities and that the highest nuclease activities can be induced when an appropriate choice is made between SpCas9-NG, SpCas9, the VRQR variant, and xCas9, the four major SpCas9 variants that have different PAM compatibilities. However, these four variants together cover only 131 (51%) or 156 (61%) out of 256 possible NNNN PAM sequences if we define a PAM as a sequence that leads to average indel frequencies higher than 10% or 5%, respectively, at the corresponding target sequences 4 days after the transduction of library A. Efficient Cas9 nucleases are not available for the remaining 49% or 39% of possible PAM sequences, necessitating the development of SpCas9 variants that have different PAM compatibilities, especially for PAM sequences that cannot be targeted using the four existing SpCas9 variants.

To overcome these restrictions in PAM compatibility, five more SpCas9 variants with wide or different PAM compatibilities have been developed since our previous high-throughput comparison; these variants include SpCas9-NRRH, SpCas9-NRTH, SpCas9-NRCH, SpG, and SpRY. In addition, Sc++, a variant of Cas9 from Streptococcus canis, has recently been proposed to have wide PAM compatibility, high on-target activity, and low off-target effects. Now, the choice of which Cas9 variant to use at a given target sequence could be particularly confusing, especially given that the PAM compatibilities of some of these variants partially overlap.

Thus, to determine the most efficient Cas9 variant at a given PAM sequence, we evaluated the activities of the four SpCas9 variants (SpCas9-NG, SpCas9, the VRQR variant, and xCas9), the five recently developed SpCas9 variants (SpCas9-NRRH, SpCas9-NRTH, SpCas9-NRCH, SpG, and SpRY), and Sc++ at 7,680 target sequences (30 sgRNAs with NNNN PAM sequences) that were previously used to determine the PAM compatibilities of SpCas9 variants²² using library A.

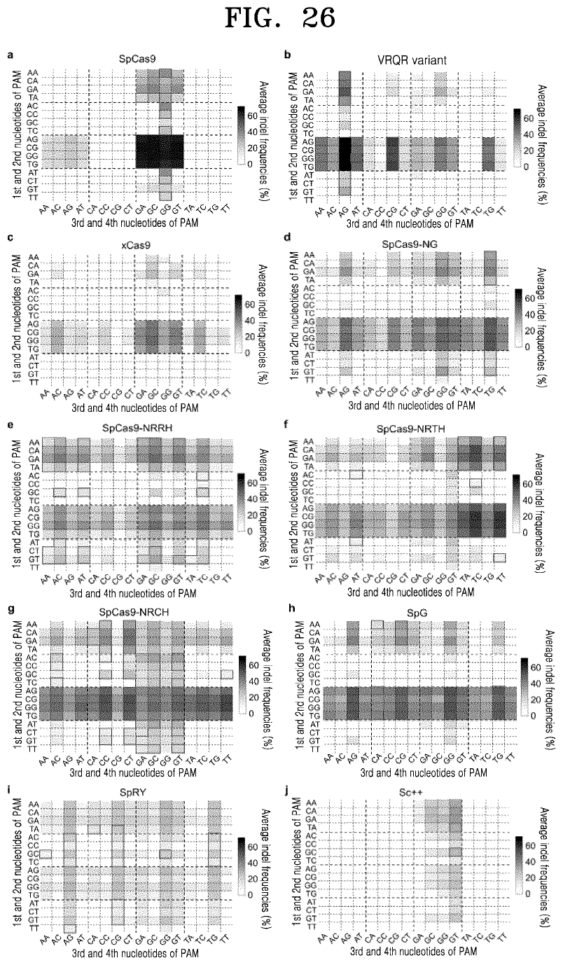

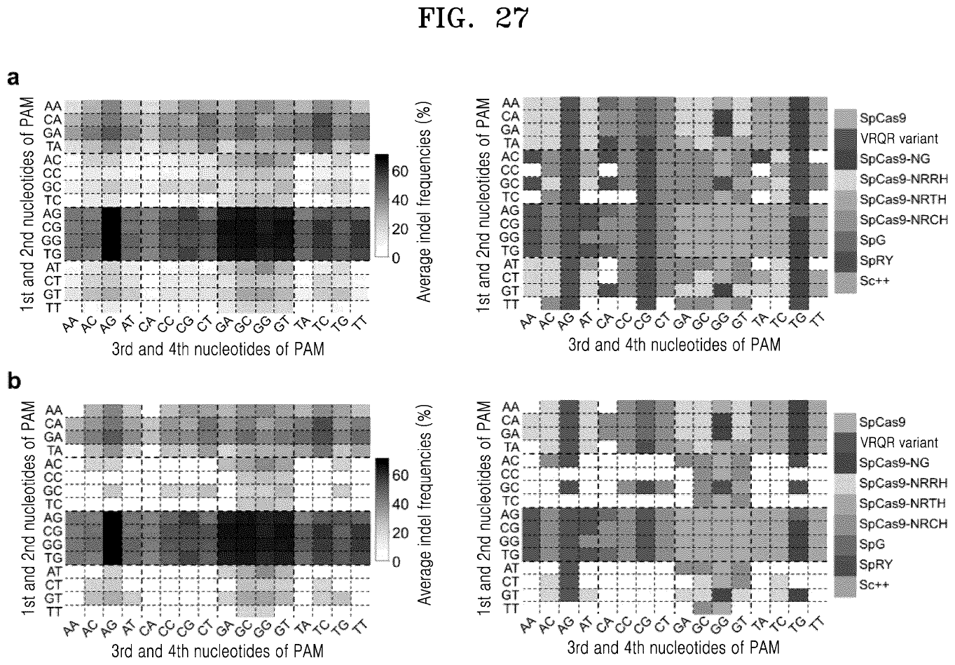

Consequently, we found that 215 out of 256 (84%) 4-nt sequences (NNNN) can be used as PAMs by at least one of the tested ten (=4+5+1) variants if we define a PAM as a sequence that leads to average indel frequencies higher than 10% at the corresponding target sequences 4 days after the transduction of library A (FIG. 19a,b and FIG. 26). If we use 5% or 20%, instead of 10%, as the criteria, then 234 (91%) or 167 (65%), respectively, out of 256 4-nt sequences were determined to be PAMs (FIG. 27). When we determined which variants exhibited the highest activity (with indel frequencies of at least 10%) at targets with each NNNN PAM sequence, SpCas9-NRCH, SpCas9-NRRH, SpCas9-NRTH, SpCas9, the VRQR variant, SpRY, SpCas9-NG, SpG, and Sc++ showed the highest activities at targets with 57, 41, 28, 25, 22, 20, 13, 7, and 2 PAM sequences, respectively. At the 84 newly added PAM sequences (=215-131), SpCas9-NRCH, SpCas9-NRRH, SpRY, SpCas9-NRTH, SpG, and Sc++ were the most efficient nucleases for 35, 24, 13, 10, 1, and 1 PAM sequences, respectively. Taken together, these results show that the addition of the recently developed variants, especially SpCas9-NRCH, SpCas9-NRRH, SpRY, and SpCas9-NRTH, substantially broadened the range of sequences that can be efficiently targeted.

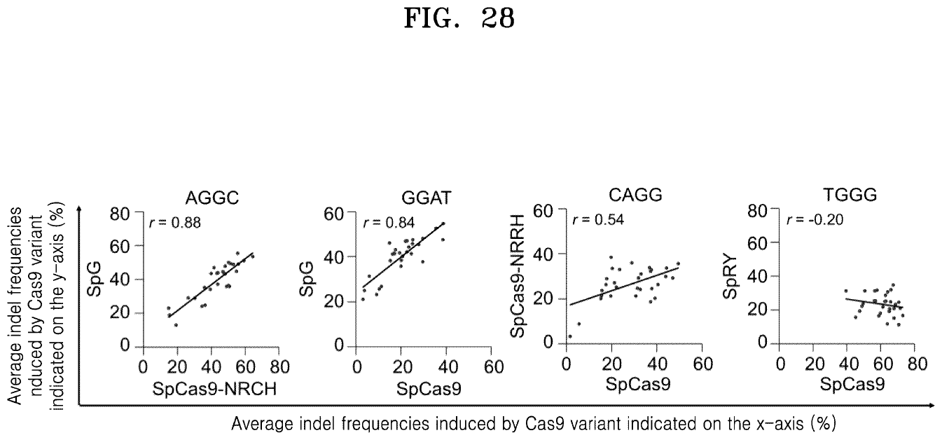

We then investigated whether the relative activities of these Cas9 variants with different PAM compatibilities were affected by the guide sequence composition at target sequences with a given shared 4-nt PAM. We found that the correlations between indel frequencies induced by the nine Cas9 variants were very diverse, with median Pearson correlation coefficients ranging from −0.20 to 0.88 (FIG. 19c and FIG. 28). The median correlations between indel frequencies from two biological replicates for the same Cas9 variant were 0.95 (for SpCas9) and 0.94 (for SpCas9-NRCH), which were higher than any median correlations between frequencies from two different Cas9 variants, suggesting that the relative activities of the Cas9 variants at sites with a given PAM sequence depend on the guide sequence composition. These correlations between the activities of SpG, the VRQR variant, SpCas9-NRCH, xCas9, and SpCas9 were relatively high, whereas those between the activities of SpRY and any of the other variants were low, sometimes even reaching negative values. When we determined the percentage of guide sequences for which the preferred Cas9 variant at a given PAM sequence showed lower activity than one of the remaining Cas9 variants at sites with the PAM by at least 1.3-fold, it ranged from 0% to 47% (mean 9.7%, median 6.7%) (FIG. 19d), suggesting that the guide sequence as well as the PAM sequence should be considered in the selection of the preferred Cas9 variant.

### Experimental Example 5. The Relationship Between the Activities of Cas9 Nucleases and Base Editors

As shown above, the window in which the highest base editing activity occurs is narrow and located at a fixed distance from the PAM. However, wild-type SpCas9 requires an NGG PAM sequence, which can theoretically be found only every 16 base pairs. Thus, efficient base editing with minimal bystander effects for a given desired edit is very frequently blocked by the lack of an NGG PAM. The utilization of Cas9 variants with different PAM compatibilities may address this problem in the use of base editors. Our results shown above provide a guide for choosing the appropriate Cas9 nuclease for a given target sequence. However, it has not been evaluated whether these conclusions from nuclease activity evaluations can be directly extrapolated to base editing, especially given that base editors include Cas9 nickase rather than Cas9 nuclease.

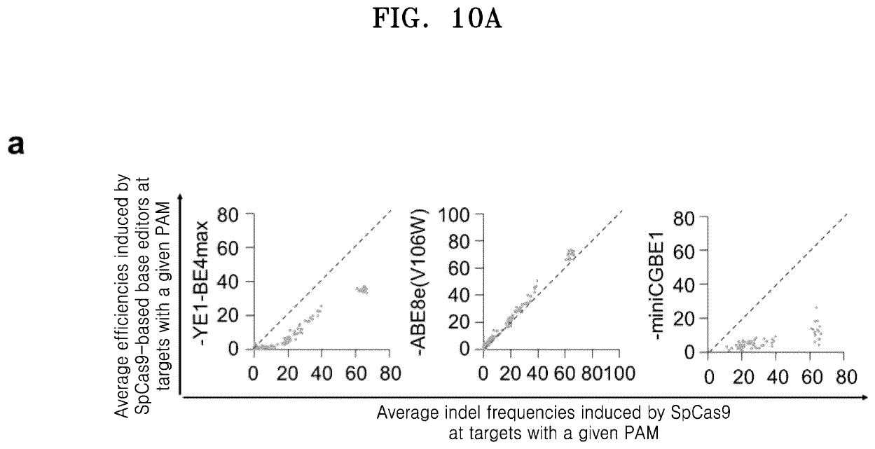

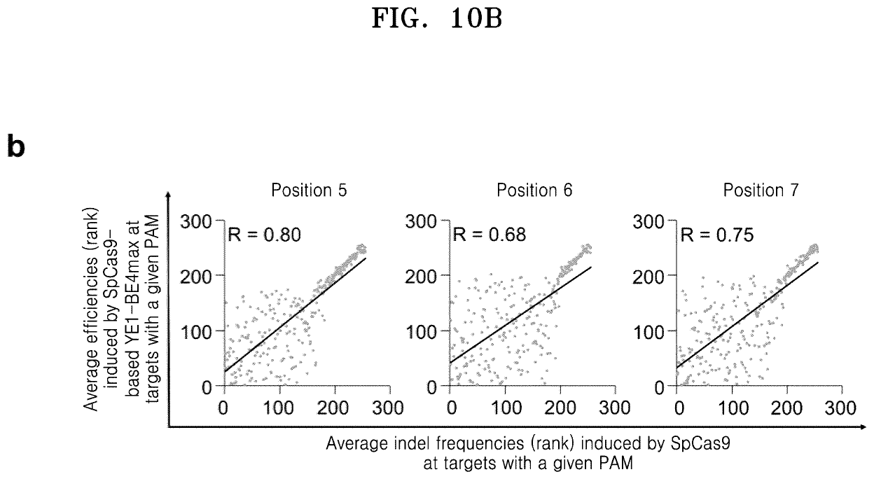

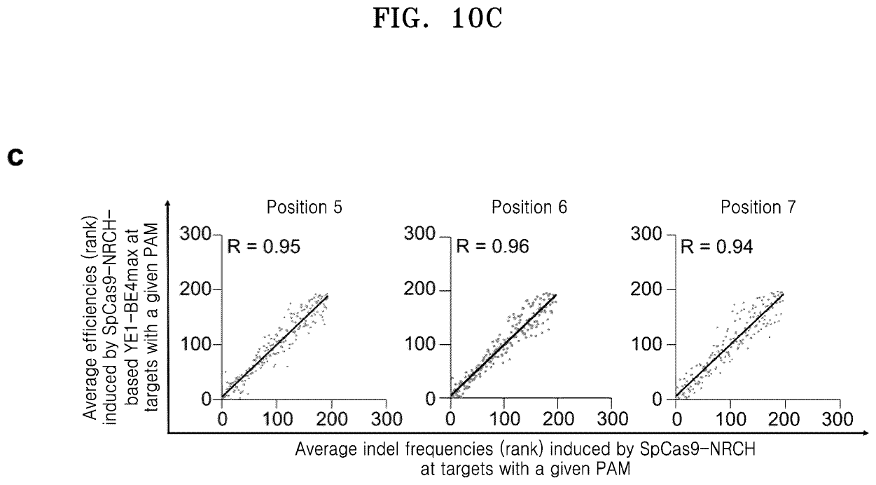

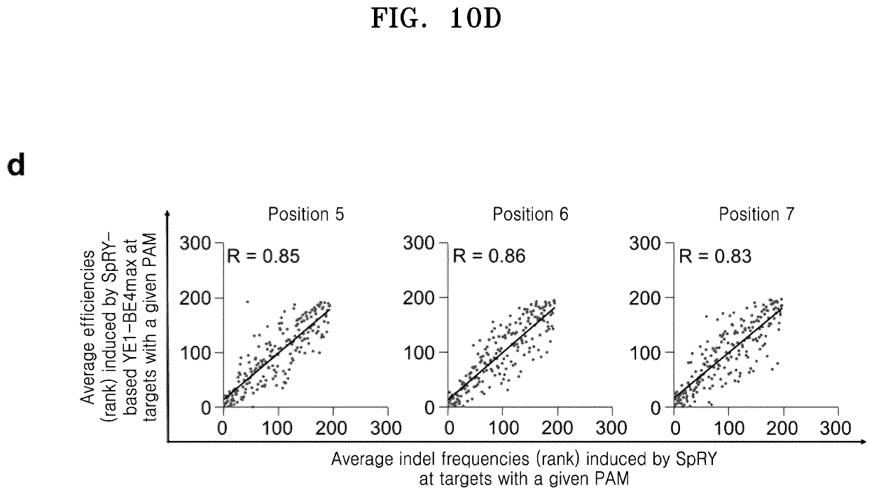

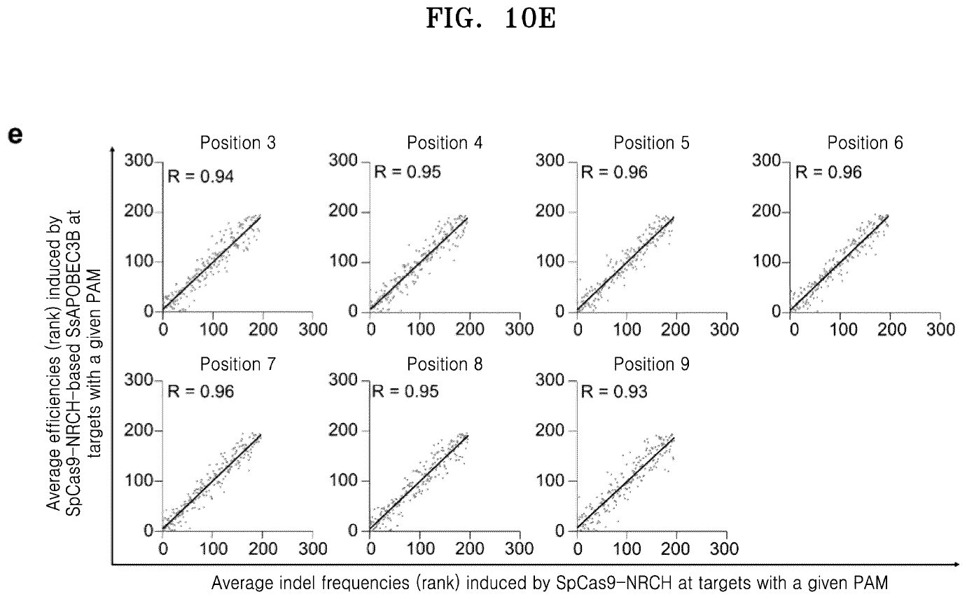

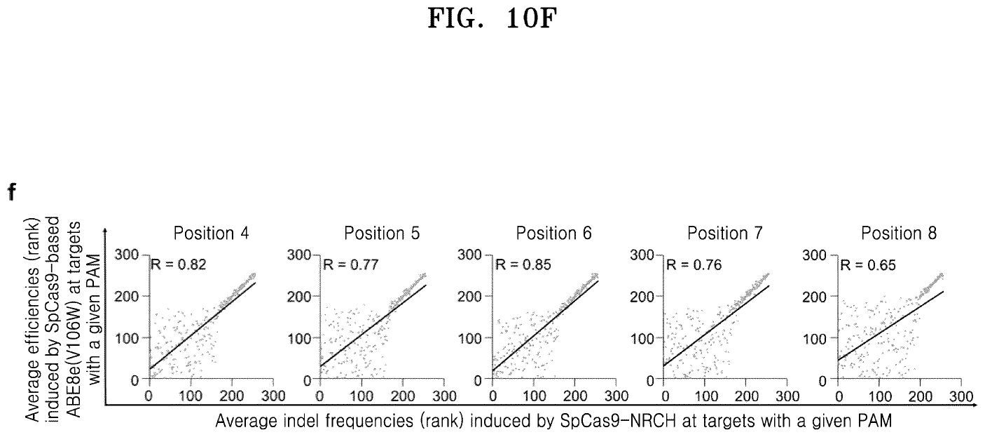

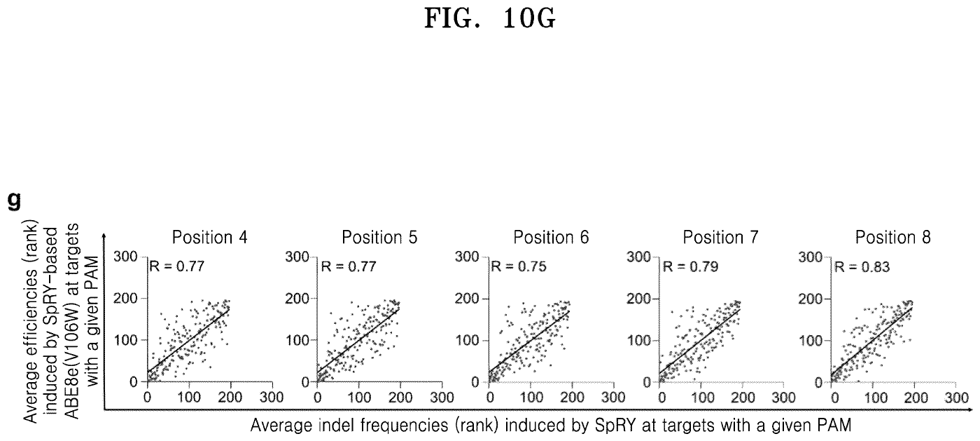

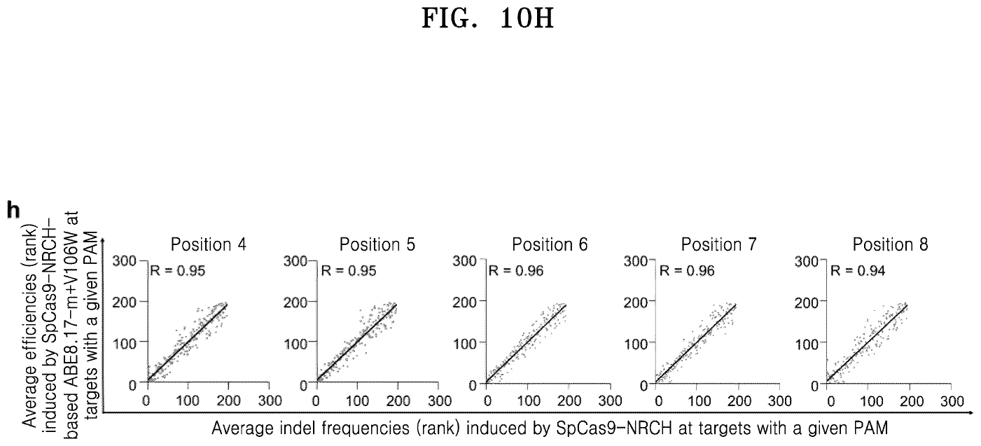

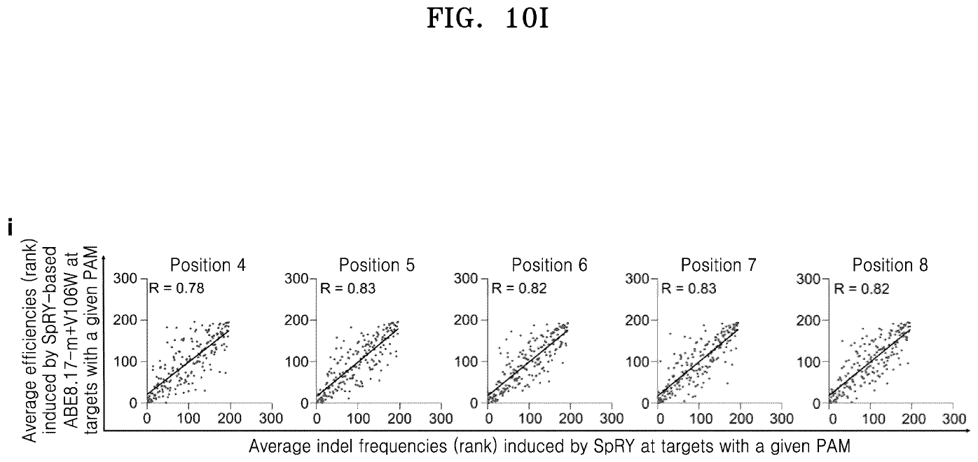

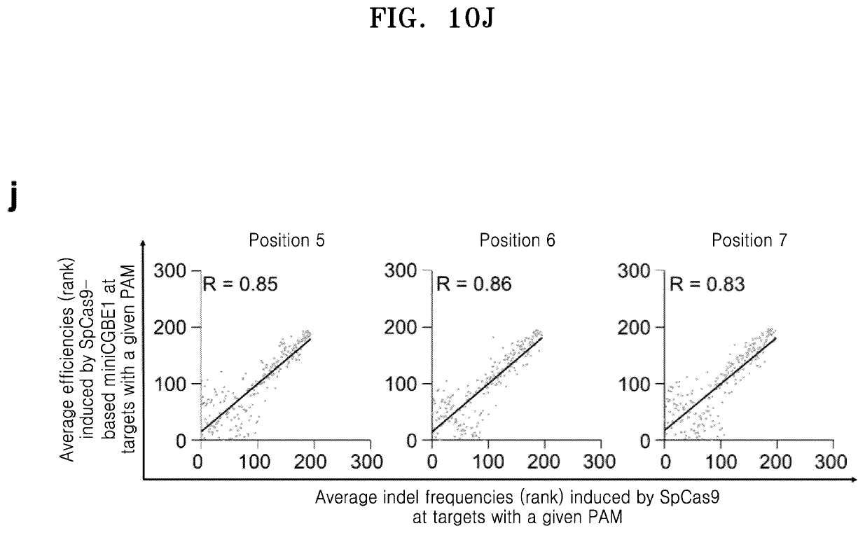

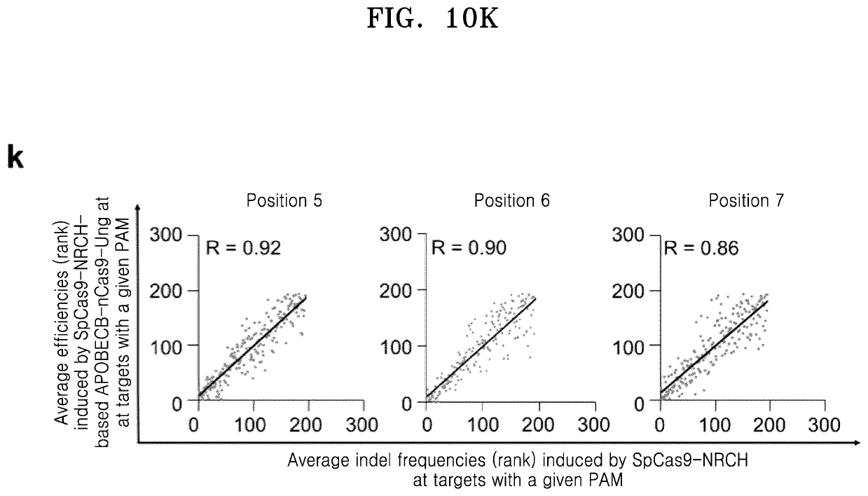

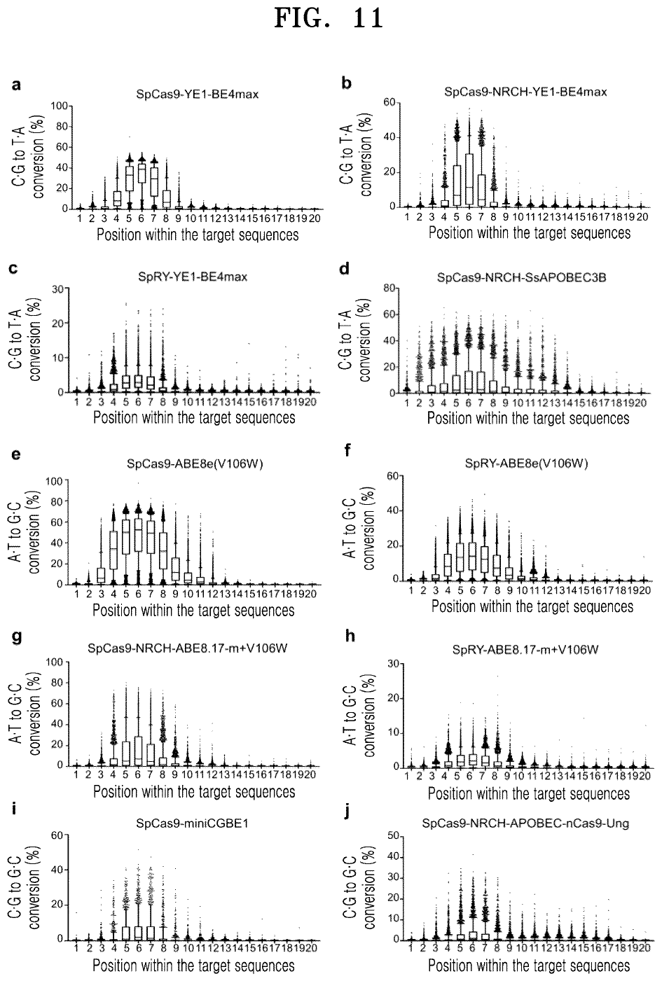

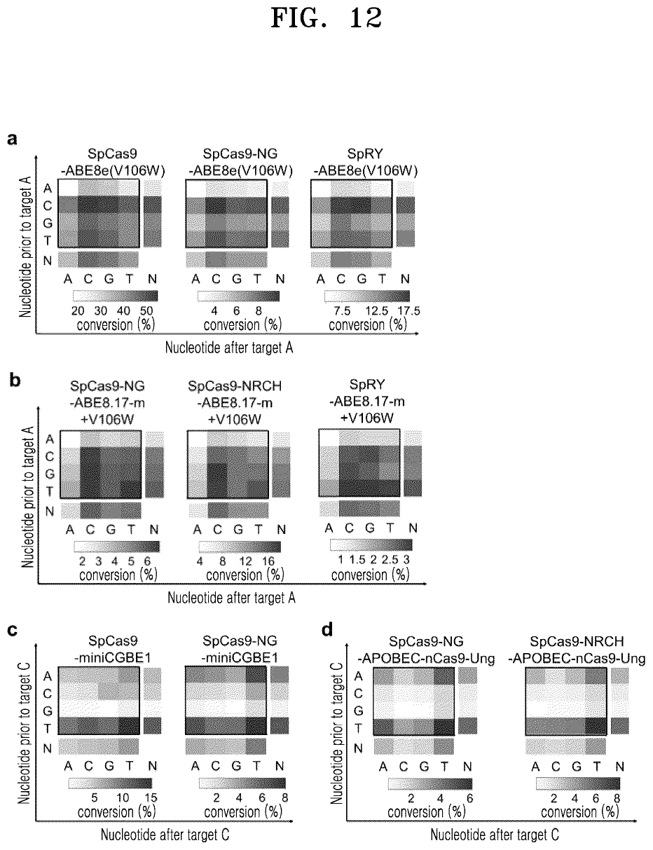

Thus, we compared the average efficiencies of base editors and Cas9 nucleases at sites with different PAM sequences using SpCas9, SpCas9-NRCH, and SpRY as example variants. As expected, the relative average efficiencies of nucleases and base editors including CBEs, ABEs, and CGBEs at sites with given PAM sequences were highly correlated (FIG. 19e,f and FIG. 10), suggesting that we can choose Cas9 nickase variants for the three classes of base editors based on the activities of Cas9 nuclease variants at sites with the PAM sequences of interest. In addition, the use of Cas9 nickase variants with different PAM compatibilities for CBEs, ABEs, and CGBEs affected overall base editing efficiencies, but did not affect the relative editing window or preferred motifs for base editing (FIG. 11,12).

### Experimental Example 6. Evaluation of SpCas9 Variants Recognizing Different PAMs, ABE8e(V106W), and YE1-BE4max Using Mismatched Target Sequences

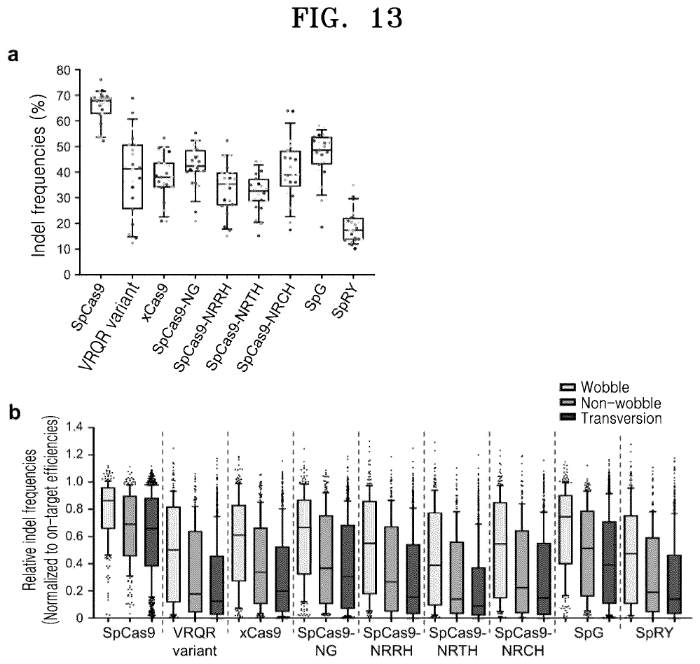

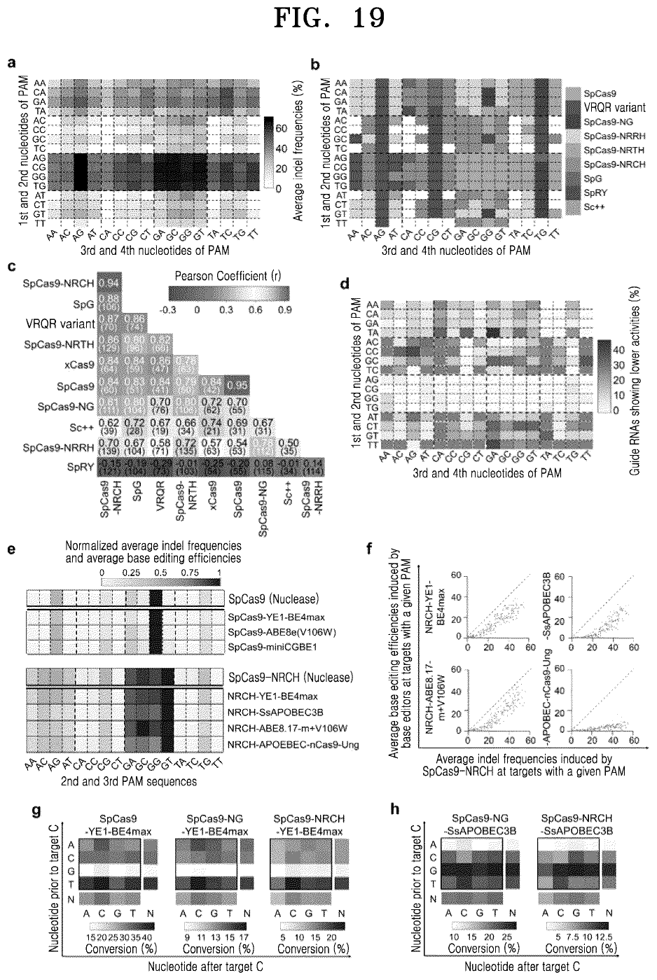

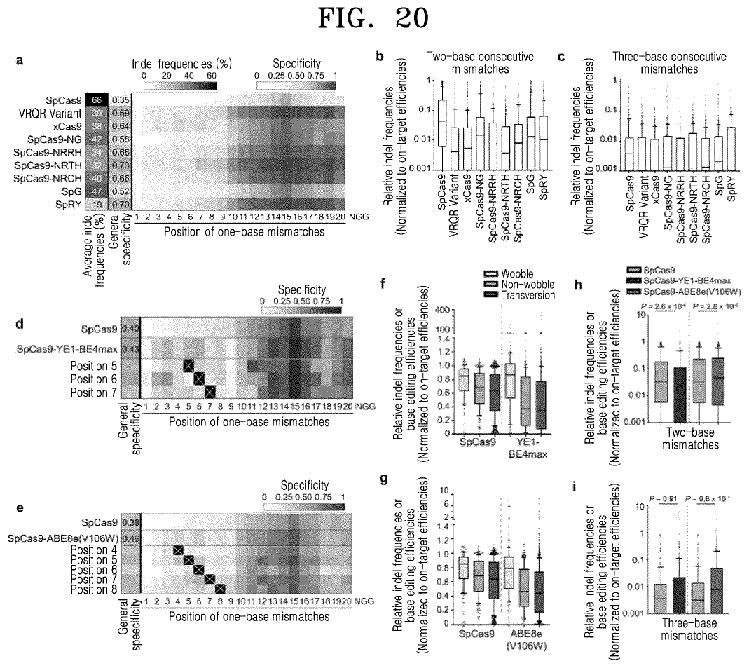

To examine the fidelity of the SpCas9 variants, we normalized the SpCas9 variant-induced indel frequencies at mismatched target sequences to those at matched targets 4 days after transduction of lentiviral Library A. For this analysis, we included in Library A 2,940 sgRNA target pairs with the following characteristics: 30 sgRNAs×98 targets (1 target without mismatches+60 targets, each with a one-base mismatch+19 targets, each with a two-base mismatch+18 targets, each with a three-base mismatch) with an NGG PAM (FIG. 20a), which was compatible with all tested Cas9 variants except for Sc++. However, the SpCas9 variants in combination with these 30 sgRNAs induced different indel frequencies at matched target sequences. If the activities at matched target sequences are drastically different between comparison groups, a comparison of activities at mismatched target sequences can be biased. Therefore, we selected 23 sgRNAs that led to relatively similar SpCas9 variant-induced indel frequencies at matched target sequences 4 days after transduction, although the average activities of SpCas9 and SpRY were higher and lower, respectively, than those of the other Cas9 variants even after this selection (FIG. 13a). When we define the specificity as 1−(indel frequencies at mismatched target sequences divided by those at perfectly matched targets), the general specificities of the variants, with the exception of SpCas9 and SpRY, were all comparable (FIG. 20a); the specificity of SpCas9 and SpRY might be relatively under- and over-estimated due to high and low indel frequencies measured at matched target sequences, respectively. The mismatch intolerance of all tested SpCas9 variants was the highest at position 15 and gradually diminished as the position became closer to 1 or to 20, with more intolerance to mismatches in the PAM-proximal regions (positions 11-20) than in the PAM-distal regions (positions 1-10). These results are in contrast to the two major peaks of intolerance to mismatches around positions 5 and 16 exhibited by some high fidelity-variants such as eSpCas9(1.1), SpCas9-HF1, HypaCas9, and evoCas9.

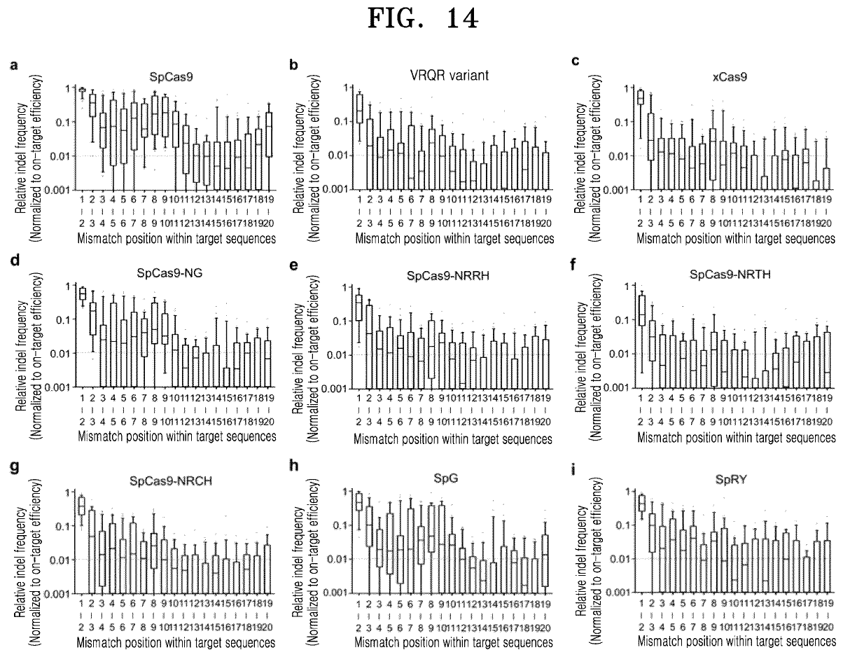

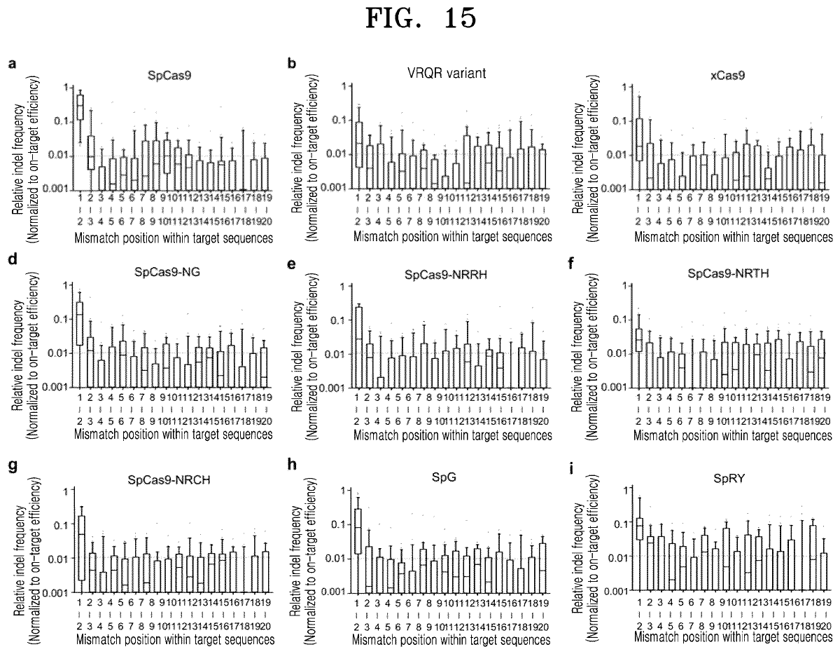

When we examined the effects of the mismatch type on mismatch tolerance, we found that all tested variants exhibited the highest tolerance at wobble transitions and the lowest at transversions (FIG. 13b), which is in line with previous results from experiments with Cas12a (or Cpf1) and SpCas9. We also found that the number of consecutive mismatched bases had a strong effect on the relative activities at mismatched targets. Consecutive two- or three-base mismatches led to a dramatic decrease in tolerance as the number of mismatches increased (FIG. 20b,c and FIG. 14, 15).

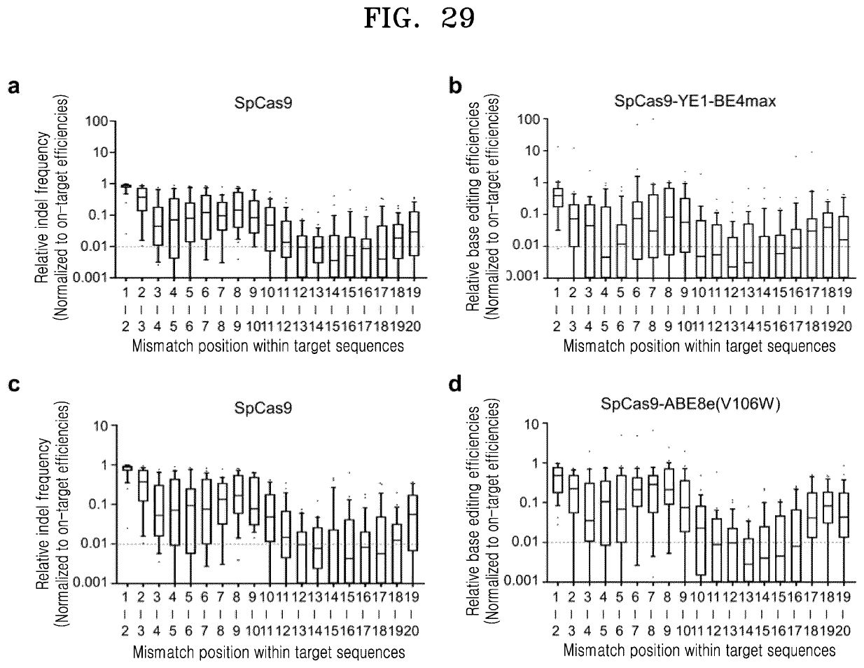

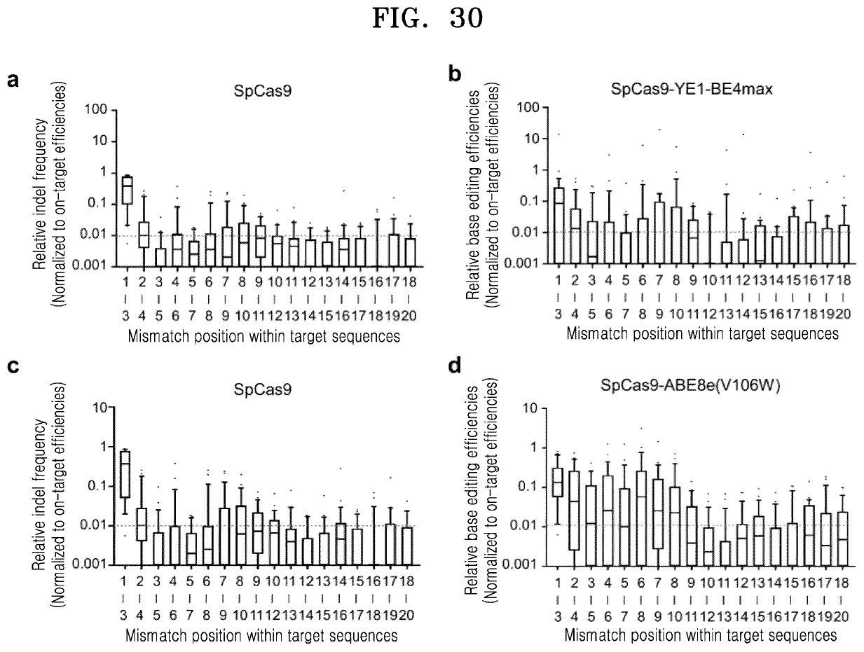

SgRNA-dependent base editor activities at mismatched target sequences have not been systemically investigated, especially in comparison with those of Cas9 nuclease. Thus, we next evaluated the fidelities of two base editors, SpCas9-YE1-BE4max and SpCas9-ABE8e(V106W), using the 2,940 sgRNA target pairs (30 sgRNAs×98 matched and mismatched targets). The general specificities of the two tested base editors were similar to those of the SpCas9 nucleases (FIG. 20d,e); as similarly observed for Cas9 nucleases, the specificity was lower in the PAM-distal region (positions 1-10) and higher in the PAM-proximal region (positions 11-20) with a peak at position 15, gradually decreasing as the position approached 1 or 20. Like Cas9 nuclease, at mismatched targets, both base editors were the most tolerant of wobble transitions and the least tolerant of transversions (FIG. 20f,g). Again similar to Cas9 nuclease, the tolerance of the base editors at mismatched targets decreased as the number of mismatches increased, as seen with consecutive two- or three-base mismatches (FIG. 20h,i and FIG. 29, 30).

### Experimental Example 7. DeepCas9variants and DeepNG-BE: Deep-Learning Based Models that Predict the Activities of Cas9 Variants and SpCas9-NG-Containing Base Editors

Because there are abundant Cas9 and base editor variants, it is currently difficult to select among them for genome editing at specific target sequences. The ability to predict the activity of each variant at target sequences of interest would be very useful in the selection of an appropriate, highly efficient variant for a specific application. To assist in this process, we first developed computational models that predict the activities of nine Cas9 variants with different PAM compatibilities-SpCas9, VRQR variant, SpCas9-NG, SpCas9-NRRH, SpCas9-NRTH, SpCas9-NRCH, SpG, SpRY, and Sc++.

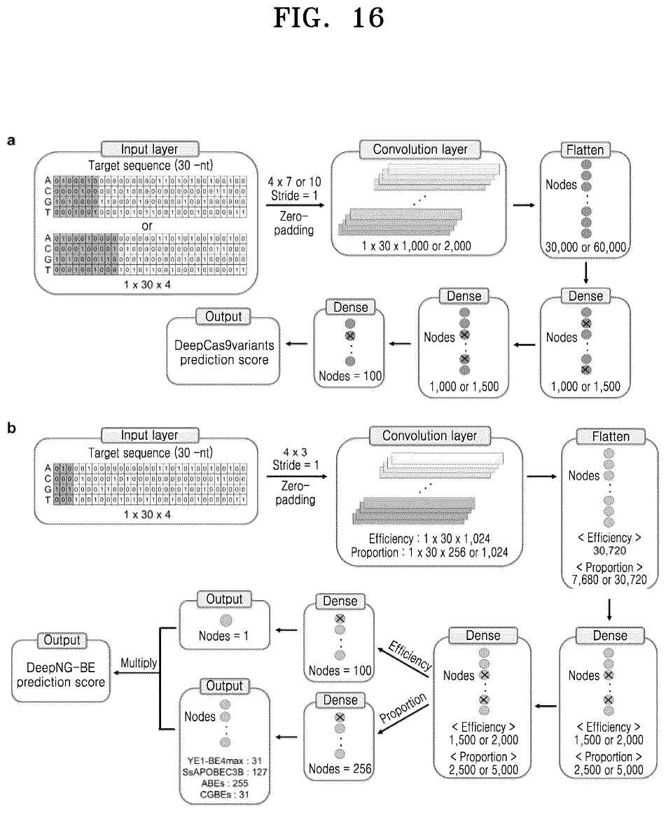

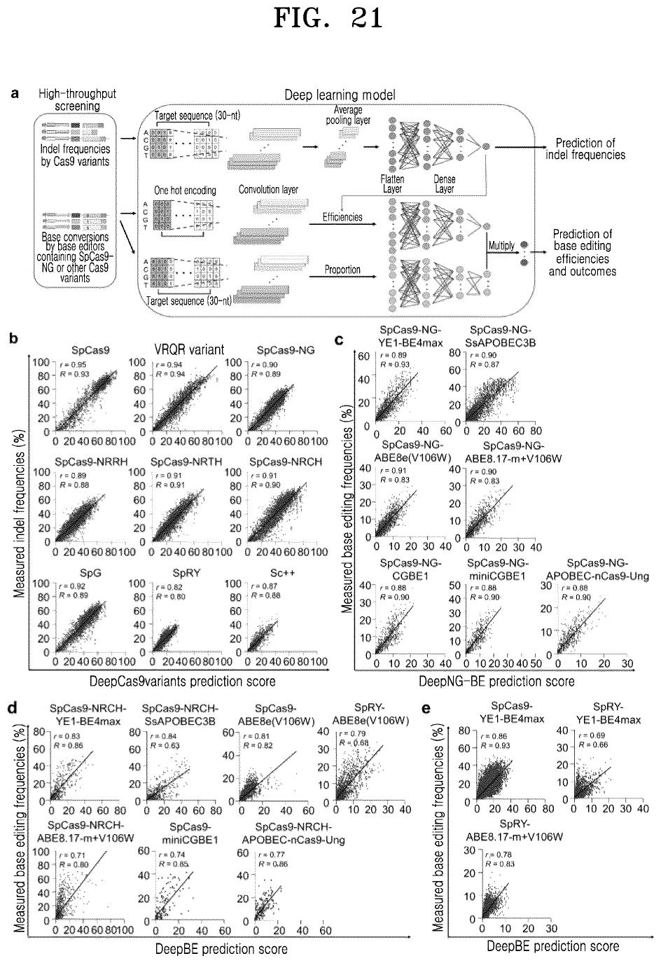

We randomly split the indel frequency data obtained for the Cas9 variants at matched target sequences with all types of PAM sequences into training and test data sets. No target sequences were shared between the training and test data sets as a result of this random splitting. We then developed, using the training data set, deep-learning-based computational models that predict the activities of the nine Cas9 variants at specified target sequences (FIG. 21a and FIG. 16a). Next, we evaluated these computational models, collectively named DeepCas9variants, with test data sets that had never been used for the training. We found that the Pearson's correlation coefficients ranged from 0.82 to 0.95 (average, 0.90), and the Spearman's correlation coefficients ranged from 0.80 to 0.94 (average, 0.89) (FIG. 21b), indicating that these models performed robustly.

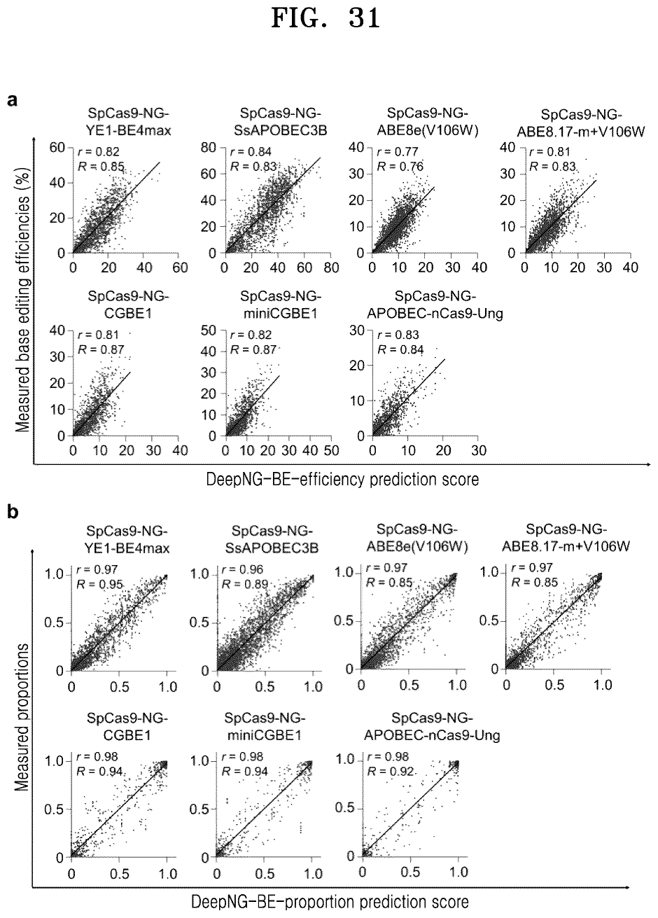

We next developed computational models that predict the editing efficiencies and outcomes of seven SpCas9-NG-containing base editors-YE1-BE4max, SsAPOBEC3B, ABE8e(V106W), ABE8.17-m+V106W, CGBE1, miniCGBE1, and APOBEC-nCas9-Ung, as we did before for previous versions of ABE and CBE. As similarly conducted for Cas9 as described above, we randomly split the base editing efficiency and outcome data from library B into training and test data sets and used the training data to produce deep-learning-based computational models, collectively named DeepNG-BE_efficiency and DeepNG-BE_proportion. These models respectively predict the base editing efficiencies and the proportions of base editing outcome sequences. Further analyses indicated that these models exhibit robust performance (FIG. 16b and FIG. 31). By combining DeepNG-BE_efficiency and DeepNG-BE_proportion, we generated computational models, collectively named DeepNG-BE, that predict the absolute frequencies of base editing outcomes. When DeepNG-BE was evaluated using test data sets that were never used for training, the Pearson's correlation coefficients ranged from 0.88 to 0.91 (average, 0.89), and the Spearman's correlation coefficients ranged from 0.83 to 0.93 (average, 0.88) (FIG. 21c), suggesting strong performance of these models.

### Experimental Example 8. DeepBE: Deep Learning-Based Models that Predict the Activities of 63 Base Editors

Using base editors that contain Cas9 variants with different PAM compatibilities as the nickase domain frequently allows the desired editing position to be located at or near the position in the base editing window at which peak editing occurs, so that the intended editing efficiency can be maximized and bystander editing effects can be minimized. Furthermore, the appropriate base-converting domain for a given base editing task can be chosen depending on the target sequence composition and the desired editing as described above. Thus, we combined the nine Cas9 variants with diverse PAM compatibilities as the nickase domain with seven different base-converting domains, generating 63 (=9×7) base editors with various PAM compatibilities. However, choosing the most appropriate base editor for an intended edit at a given target sequence would be particularly difficult when there are so many choices. Thus, we next attempted to develop computational models that predict base editing efficiencies and outcomes for the 63 base editors at a given target sequence. However, measuring the efficiencies of all 63 base editors at a large number of target sequences would be extremely time consuming and costly.

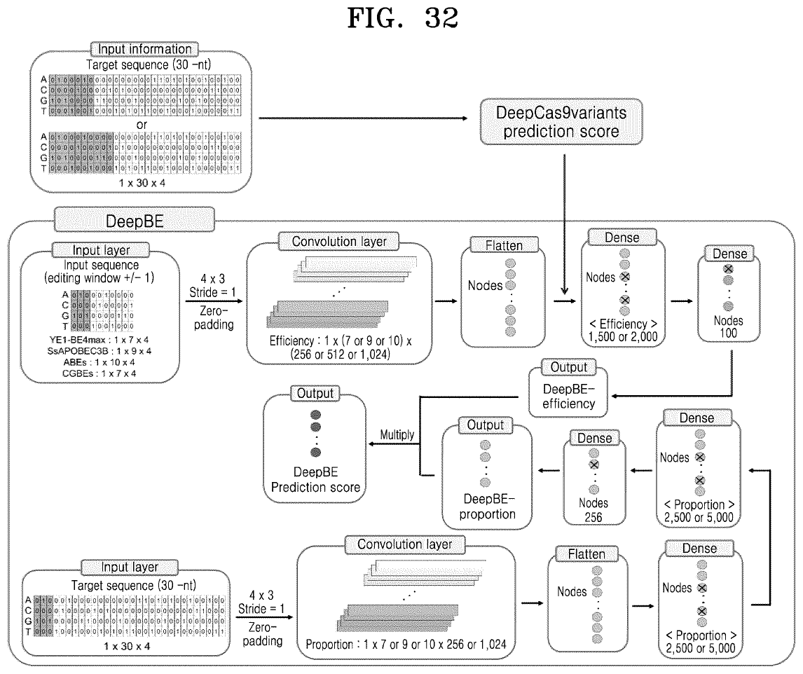

Given that base editing efficiencies would be affected by both the target nucleotide converting activity and the Cas9 nickase activity, we postulated that deep learning using factors that affect the base-converting activity and the Cas9 activity as the input information could enable prediction of base editing efficiency. Sequence motifs surrounding the target nucleotide affect base editing as shown in this and previous studies and different deaminases often have different preferred motifs. Thus, to reflect base-converting activity for the seven types of base editors with different base-converting domains, we used editing windows±1 nucleotide as input information, which would mainly affect base-converting activity rather than Cas9 nickase activity (FIG. 32). Furthermore, as additional input information, we used DeepCas9 variant scores, which reflect Cas9 activity. As the training data sets, we used base editing efficiency data generated using seven SpCas9-NG-containing base editors (YE1-BE4max, SsAPOBEC3B, ABE8e(V106W), ABE8.17-m+V106W, CGBE1, miniCGBE1, and APOBEC-nCas9-Ung) and seven base editors containing diverse Cas9 nickase variants (i.e., SpCas9-NRCH-YE1-BE4max, SpCas9-NRCH-SsAPOBEC3B, SpCas9-ABE8e(V106W), SpRY-ABE8e(V106W), SpCas9-NRCH-ABE8.17-m+V106W, SpCas9-miniCGBE1, and SpCas9-NRCH-APOBEC-nCas9-Ung).

As a result of this process, we developed DeepBE_efficiency, which predicts the efficiencies of 63 base editors. To predict the relative proportions of base editing outcomes, we used DeepNG-BE_proportion, given that the relative proportions of base editing outcomes will be determined by the base-converting activity and guide sequence rather than the PAM sequence. By combining the predicted results of DeepBE_efficiency and DeepNG-BE_proportion, we developed DeepBE, which predicts the absolute outcome frequencies of base editing for the 63 base editors. When we tested DeepBE for seven base editors containing diverse Cas9 nickase variants (which were used to generate the training data sets) with test target sequences that were never used for training, we found that the Pearson's correlation coefficients ranged from 0.72 to 0.84 (average, 0.78), and the Spearman's correlation coefficients ranged from 0.63 to 0.86 (average, 0.79) (FIG. 21d), suggesting good performance of these models. Furthermore, when we tested DeepBE for three base editors containing diverse Cas9 nickase variants that were not used to generate the training data sets with test target sequences that were never used for training, the Pearson's correlation coefficients ranged from 0.69 to 0.86 (average, 0.78), and the Spearman's correlation coefficients ranged from 0.66 to 0.93 (average, 0.81) (FIG. 21e), indicating the good generalization performance of DeepBE. We have provided these models as web tools at http://deepcrispr.info/DeepBE. Researchers can use the tools to select the most appropriate base editor variant and sgRNA pair to obtain their desired edit efficiently at target sequences of interest.

### Experimental Example 9. Choosing the Most Efficient Base Editor Variant and Guide Sequence Pair for Correcting Pathogenic or Likely Pathogenic Mutations

Among 75,104 pathogenic or likely pathogenic mutations reported in ClinVar, 5,475 (7.3%), 15,040 (20%), and 4,492 (6.0%) of them can be corrected by C•G to T•A, A•T to G•C, and C•G to G•C editing, respectively. C•G to T•A editing can be induced using 18 CBE variants (=two base-converting domains×nine Cas9 nickase variants with different PAM compatibilities). Similarly, A•T to G•C editing and C•G to G•C editing can be generated using 18 ABE variants and 27 CGBE variants, respectively. However, choosing the best base editor variant and sgRNA pair for achieving the maximum frequency of intended edits is not easy. Given that the base editing windows for CBEs and ABEs are 5-bp wide (although SSsAPOBEC3B has a wider (7-bp) editing window, we considered only 5-bp editing windows spanning positions 4-8 so that fair comparisons could be made) and those for CGBEs are 3-bp wide, there are 18×5=90 theoretically possible guide sequence and base editor pairs for C•G to T•A and A•T to G•C editing and 27×3=81 pairs for C•G to G•C editing.

An efficient pair could be chosen rationally. First, we could conduct SpCas9-based rational design, in which SpCas9 is chosen as the Cas9 nickase domain. Using this approach, we designed guide sequences so that the editing positions for CBEs were located at positions 6, 7, 5, 4, or 8 (in order of preference), those for ABEs at positions 6, 5, 7, 4, or 8, and those for CGBEs at positions 6, 5, or 7, in each case determining whether an NGG PAM sequence was located at the appropriate position. If these processes did not identify any position that allowed for an NGG PAM, we then located the intended edit at position 6 and selected SpCas9 regardless of the PAM sequence. In another form of rational design, which we call Cas9 variant-based design, we first located the intended edit at position 6 and then choose a Cas9 variant that recognized a PAM at the appropriate position using the information shown in FIG. 19b. If none of the available Cas9 variants recognized the PAM, then we located the editing position for CBEs at positions 7, 5, 4, or 8, those for ABEs at positions 5, 7, 4, or 8, and those for CGBEs at positions 5 or 7 until a PAM recognized by a Cas9 variant was identified. If these processes did not lead to the identification of an appropriate Cas9 variant, we located the intended edit at position 6 and selected SpCas9-NRCH, the variant that showed the widest PAM compatibility. Once the guide sequence and Cas9 domain are determined, then the base-converting domain can be chosen either randomly or by selecting the base-converting domain that showed relatively higher overall editing efficiencies (i.e., SsAPOBEC3B, ABE8e(V106W), and CGBE1 as the CBE, ABE, and CGBE, respectively). Alternatively, one can randomly choose the SpCas9 variant and base-converting domains so that the editing position is located at position 6 (Random design). Furthermore, we can predict the editing efficiencies and outcomes of 90 or 81 guide sequence and base editor pairs using DeepBE and choose the most efficient one (DeepBE-based design).

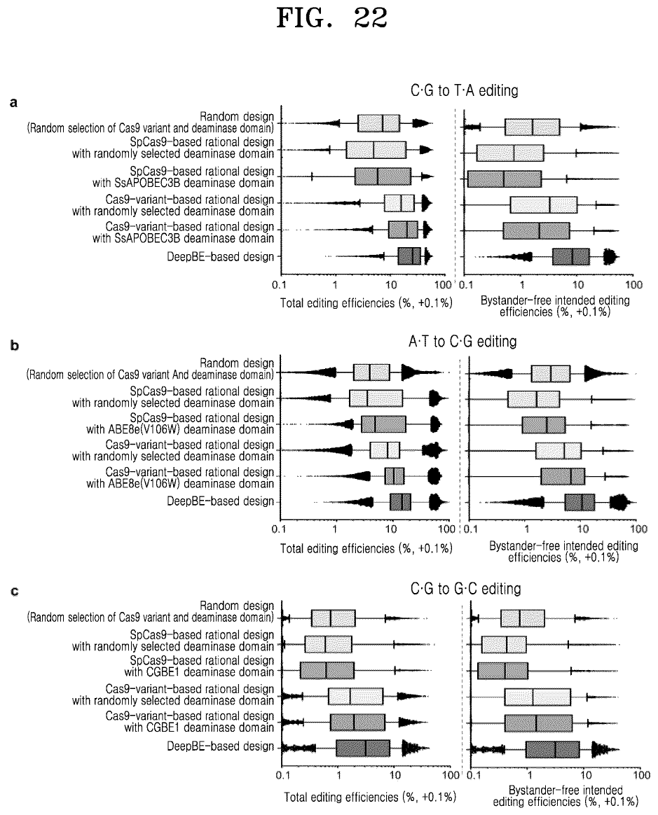

When we compared the predicted efficiencies of base editing and intended editing without bystander editing using these two forms of SpCas9-based rational design, two forms of Cas9 variant-based rational design, a random design, and a DeepBE-based design, the DeepBE-based design showed substantially higher expected editing efficiencies as compared to the other approaches for both total intended base editing and bystander editing-free intended editing for all three types of editing (i.e., C•G to T•A, A•T to G•C, and C•G to G•C editing) (FIG. 22a-c). If DeepBE-based design is not considered, the best design methods are as follows. For total and bystander-free C•G to T•A editing, Cas9 variant-based rational design using SsAPOBE3B and that using a randomly selected base-converting domain, respectively, showed the highest median efficiencies. For both total and bystander-free A•T to G•C editing, Cas9 variant-based rational design using ABE8e(V106W) showed the highest median efficiencies. For total and bystander-free C•G to G•C editing, Cas9 variant-based rational design using the CGBE1 base-converting domain showed the highest median efficiencies. When median efficiencies associated with these Cas9 variant-based designs were compared with those associated with the corresponding SpCas9-based designs, the fold increases were 3.5-, 2.2-, and 3.5-fold for total C•G to T•A, A•T to G•C, and C•G to G•C editing, respectively, and 4.8-, 2.8-, and 4.4-fold for bystander-free C•G to T•A, A•T to G•C, and C•G to G•C editing, respectively. When the median efficiencies associated with DeepBE-based design were compared with those associated with SpCas9-based designs, the fold increases were 4.4-, 3.0-, and 5.8-fold for total C•G to T•A, A•T to G•C, and C•G to G•C editing, respectively, and 12-, 4.4-, and 9.9-fold for bystander-free C•G to T•A, A•T to G•C, and C•G to G•C editing, respectively. Taken together, these findings indicate that both Cas9 variant-containing base editors and DeepBE can substantially improve desired base editing efficiencies.

The above descriptions of the disclosure is provided only for illustrative purposes, and those of skill in the art will understand that the disclosure may be easily modified into other detailed configurations without modifying technical aspects and essential features of the disclosure. Hence, it should be understood that the above-described embodiments are not limiting of the scope of the disclosure.

According to the system for predicting the efficiency and an outcome of a base editor by using deep learning according to one aspect, it is possible to select a base editor from among 63 base editors with various PAM compatibilities and sgRNA for efficient base editing, without extensive experiments. Therefore, the system may be usefully used in all fields where gene editing is applied, such as disease treatment by gene editing.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A system for predicting efficiency and an outcome of a base editor by using deep learning, the system comprising:
a target sequence input unit configured to receive an input of target sequence data of the base editor; and
an outcome prediction unit configured to obtain a base editing efficiency output value and a base editing outcome proportion output value by applying the target sequence data that is input through the target sequence input unit, to a base editing efficiency prediction model and a base editing outcome proportion prediction model, respectively, and generate a base editing prediction score by multiplying the base editing efficiency output value by the base editing outcome proportion output value.

2. The system of claim 1, wherein the base editing efficiency prediction model is generated by:
receiving an input of base conversion activity data of the base editor through an information input unit; and
generating the base editing efficiency prediction model by performing deep learning based on a convolutional neural network (CNN) on the base conversion activity data that is input through the information input unit.

3. The system of claim 2, wherein the generating of the base editing efficiency prediction model by performing the deep learning based on the CNN further comprises linking CRISPR associated protein 9 (Cas9) activity data.

4. The system of claim 3, wherein the Cas9 activity data is obtained by performing a method comprising:
introducing Cas9 into a cell library containing oligonucleotides containing a nucleotide sequence that encodes sgRNA and a target nucleotide sequence targeted by the sgRNA;
performing deep sequencing by using DNA obtained from the cell library into which the Cas9 is introduced; and
analyzing efficiency of the Cas9 based on data obtained from the deep sequencing.

5. The system of claim 4, wherein the analyzing of the efficiency of the Cas9 comprises predicting an activity of the Cas9 based on a correlation between indel frequencies of the Cas9 in a particular target sequence by performing deep learning based on a CNN.

6. The system of claim 1, wherein the base editing outcome proportion prediction model is generated by:
receiving an input of base editing outcome data of the base editor through an information input unit; and
generating the base editing outcome proportion prediction model by performing deep learning based on a CNN on the base editing outcome data that is input through the information input unit.

7. The system of claim 1, further comprising an output unit configured to output efficiency and an outcome proportion of the base editor, which are predicted by the outcome prediction unit.

8. The system of claim 3, wherein the Cas9 is any one or more selected from a group consisting of SpCas9, VRQR variant, SpCas9-NG, SpCas9-NRRH, SpCas9-NRTH, SpCas9-NRCH, SpG, SpRY, and Sc++.

9. The system of claim 1, wherein the base editor is any one or more selected from a group consisting of YE1-BE4max, SsAPOBEC3B, ABE8e(V106W), ABE8.17-m+V106W, CGBE1, miniCGBE1, and APOBEC-nCas9-Ung.

10. The system of claim 1, wherein the base editing efficiency output value is calculated through Equation 1 below:
$$Base editing efficiency ( \% ) = \frac{\begin{matrix}Total read counts of intended target \\ nucleotide conversions at each position\end{matrix}}{Total read counts} \times 100 \qquad [ Equation 1 ]$$

11. The system of claim 1, wherein the base editing outcome proportion output value is calculated through Equation 2 below:
$$Base editing outcome proportion = \frac{\begin{matrix}Total read counts of unique \\ base - edited outcome sequence\end{matrix}}{\begin{matrix}Total read counts of converted \\ sequences within wide windows\end{matrix}} \qquad [ Equation 2 ]$$

12. A method of predicting efficiency and an outcome of a base editor by using deep learning, the method comprising:
designing a target sequence of the base editor; and
applying the designed target sequence to the system for predicting efficiency and an outcome of a base editor of claim 1.

13. A computer-readable recording medium having recorded thereon a program for causing a computer to execute the method of claim 12.
